(19) United States
(12) Patent Application Publication
(10) Pub. No.: US 2010/0099084 A1
(43) Pub. Date: Apr. 22, 2010

(54) DETECTION OF NPM1 NUCLEIC ACID IN ACELLULAR BODY FLUIDS

(76) Inventor: Maher Albitar, Coto De Caza, CA (US)

Correspondence Address: FOLEY & LARDNER LLP, P.O. BOX 80278, SAN DIEGO, CA, 92138-0278, US

(21) Appl. No.: 12/336,475

(22) Filed: Dec. 16, 2008

Related U.S. Application Data
(60) Provisional application No. 61/106,532, filed on Oct. 17, 2008.
(60) Provisional application No. 61/110,941, filed on Nov. 3, 2008.

(51) Int. Cl. C12Q 1/68

(52) U.S. Cl. 435/6

(57) ABSTRACT

The present inventions relates to methods for detecting NPM1 nucleic acid in acellular body fluid samples and determining whether the nucleic acid contains one or more mutations including insertions and deletions. The methods are useful for predicting prognosis of AML patients that have cells with mutations in the NPM1 gene.

### CROSS-REFERENCE TO RELATED APPLICATIONS

The application claims the benefit of U.S. Provisional Applications 61/106,532, filed Oct. 17, 2008 and 61/110,941, filed Nov. 3, 2008, each of which is hereby incorporated by reference in its entirety.

### FIELD OF THE INVENTION

The disclosed inventions relate to the field of oncology, including cancer diagnosis and therapy.

### BACKGROUND OF THE INVENTION

The following discussion of the background of the invention is merely provided to aid the reader in understanding the invention and is not admitted to describe or constitute prior art to the invention.

Nucleophosmin also known as B23, numatrin, and NO38, is a ubiquitously expressed nucleolar phoshoprotein which shuttles continuously between the nucleus and cytoplasm. Nucleophosmin functions include binding of nucleic acids, regulation of centrosome duplication and ribosomal function, and regulation of the ARF-p53 tumor suppressor pathway.

The gene encoding Nucleophosmin is NPM1. The NPM1 gene is located on chromosome 5q35. Disruption of NPM1 by reciprocal chromosomal translocation is involved in several hematolymphoid malignancies (Falini et al. Hematologica. 2007; 92(4): 519-532). These translocations result in the formation of various fusion proteins that retain the N-terminus of Nucleophosmin and have been associated with neoplastic conditions including NPM-anaplastic large cell lymphoma kinase (NPM-ALK) in anaplastic large cell lymphoma (Morris et al. Science. 1994; 263:1281-1284), NPM-retinoic acid receptor-alpha (NPM-RARα) in acute promyelocytic leukemia (Redner et al. Blood. 1996; 87: 882-88), and NPM-myelodysplasia/myeloid leukemia factor 1 (NPM-MLF1) in AML/myclodysplastic syndrome (Yoneda-Kato et al. Oncogene. 1996;12: 265-275).

Heterozygous mutations of the NPM1 gene have been identified in approximately 35% of adult patients as well as 6.5% of children with acute myeloid leukemia (AML) (Falini et al. N. Engl. J. Med. 2005; 352: 254-266; Cazzaniga et al. Blood. 2005; 106:1419-1422). Many molecular variants of NPM1 mutations have been described to date in AML patients, with the majority falling in exon 12 (Falini et al. Blood. 2007; 109: 874-85). Many of the NPM1 mutations that have been identified in AML are characterized by simple 1- or 2-tetranucleotide insertions, a 4-base pair (bp) or 5-bp deletion combined with a 9-bp insertion, or a 9-bp deletion combined with a 14-bp insertion (Falini et al. Blood. 2007;109: 874-85; Chen et al. Arch. Pathol. Lab Med. 2006; 130: 1687-1692). Mutations in exon 12 of the NPM1 gene often lead to frame shifts, generating an elongated protein which is retained in the cytoplasm.

NPM1 mutations are associated with high levels of bone marrow blasts, a high white blood cell (WBC) and platelet count, and fms-related tyrosine kinase 3 internal tandem duplication (FLT3-ITD) (Thiede et al. Blood. 2006; 107: 4011-4020). Patients exhibiting NPM1 mutations without FLT3 mutations showed significantly better overall and disease-free survival in this study (Thiede et al. Blood. 2006; 107: 4011-4020). NPM1 mutations are common in AML with a normal karyotype (Schnittger et al. Blood. 2005; 106: 3733-3739). Within the group of patients with AML who have a normal karyotype, various studies have shown that patients with NPM1-mutated AML had a complete remission rate similar to or significantly higher than that of patients with wild-type NPM1 AML (Boissel et al. Blood. 2005; 106: 3618-3620; Falini et al. N. Engl. J. Med. 2005; 352: 254 266; Suzuki et al. Blood. 2005; 106: 2854-2861; Dohner et al. Blood. 2005; 106: 3740-6).

### SUMMARY OF THE INVENTION

The present invention provides methods for the detection of NPM1 nucleic acid in an acellular body fluid. In certain aspects, the invention including determining whether the NPM1 nucleic acid comprises one or more mutations. The invention also provides methods for determining a diagnosis or prognosis of an individual diagnosed as having AML, based on determining the presence or absence of NPM1 gene mutation(s).

In one aspect, the invention provides methods for detecting the presence or absence of NPM1 nucleic acid in an acellular body fluid of an individual. The individual may be diagnosed as having a malignant disorder (e.g., AML or MDS), or may be suspected of developing one.

In another aspect, the invention provides a method of determining a prognosis of an individual diagnosed with a hematologic disorder (e.g., AML or MDS), comprising determining the presence or absence of one or more mutations in an NPM1 nucleic acid, wherein the NPM1 nucleic acid is obtained from an acellular body fluid of the individual, and providing a prognosis for said individual, wherein the presence of one or more mutations in the NPM1 gene is indicative of better prognosis for the individual relative to an individual diagnosed with AML and lacking the one or more mutations. Suitable acellular body fluid include, for example, serum and plasma. Suitable NPM1 nucleic acids that are isolated and/or assessed include, for example, genomic DNA and RNA (e.g., mRNA).

In preferred embodiments, the NPM1 mutations are determined relative to the NPM1 sequence of SEQ ID NO: 1. In some embodiments, one or more of the NPM1 mutations assessed is selected from the mutations in FIG. 2A or 2B. In other embodiments, the NPM1 mutation is an insertion mutation including, for example, an insertion after the nucleotide corresponding to position 1018 of SEQ ID NO: 1. In other embodiments, the insertion is a CTCT or a CTCG insertion. The presence of an NPM1 mutation, including an insertion mutation, is associated with an improved prognosis (i.e., a better prognosis than an individual diagnosed with the hematological disorder and lacking the NPM1 mutation). In preferred embodiments, the improved prognosis is an improved remission rate or an improved overall survival rate relative to an individual diagnosed as having a hematologic disorder but lacking an NPM1 mutation.

In other embodiments, the nucleic acid obtained from the acellular body fluid is further assessed for the presence or absence of one or more mutations in the FLT3 gene. In some embodiments, the FLT3 gene mutation is a duplication of an internal tandem repeat. Under one interpretation, an individual lacking an FLT3 mutation and further containing an NPM1 mutation has an improved prognosis relative to an individual diagnosed as having a hematological disorder and either or both of an NPM1 mutation and an FLT3 mutation.

In other embodiments, the method further comprises determining the cytogenetics of the individual. Under one interpretation, an individual having intermediate cytogenetics and further comprising an NPM1 mutation has an improved prognosis relative to an individual lacking an NPM1 mutation and having intermediate, normal, or poor cytogenetics. In another interpretation, an individual having normal cytogenetics and further comprising an NPM1 mutation has an improved prognosis relative to an individual having normal cytogenetics and lacking an NPM1 mutation.

In other embodiments, the presence or absence of an NPM1 mutation is assessed by determining the nucleotide sequence of at least a portion of the NPM1 nucleic acid. In another embodiment, the presence or absence of an NPM1 mutation is assessed by determining the size of at least a portion of the NPM1 nucleic acid. Optionally, the NPM1 nucleic acid is amplified. Amplification may be performed using oligonucleotide amplification primers of SEQ ID NO: 3 and/or SEQ ID NO: 4. Optionally, the zygosity status of the individual is determined.

In another aspect, the invention provides a method for diagnosing an individual as having a hematological disorder by determining the presence or absence of a translocation in an NPM1 nucleic acid obtained from an acellular body fluid, and diagnosing said individual with a hematological disorder when a translocation in an NPM1 nucleic acid is detected. In certain embodiments, the hematological disorder is anaplastic large cell lymphoma, acute promyelocytic leukemia, and acute myelogenous leukemia. In other embodiments, the translocation occurs between the NPM1 gene and one of the anaplastic large cell lymphoma kinase, retinoic acid receptor-alpha, or myelodysplasia/myeloid leukemia factor 1 genes. Optionally, the individual may be further assessed for one or more mutations in the NPM1 gene and/or the FLT3 gene, as described for the foregoing aspects.

The term “sample” or “patient sample” as used herein includes biological samples such as tissues and bodily fluids. “Bodily fluids” may include, but are not limited to, blood, serum, plasma, saliva, cerebral spinal fluid, pleural fluid, tears, lactal duct fluid, lymph, sputum, urine, amniotic fluid, and semen. A sample may include a bodily fluid that is “acellular.” An “acellular bodily fluid” includes less than about 1% (w/w) whole cellular material. Plasma or serum are examples of acellular bodily fluids. A sample may include a specimen of natural or synthetic origin (i.e., a cellular sample made to be acellular).

“Plasma” as used herein refers to acellular fluid found in blood. “Plasma” may be obtained from blood by removing whole cellular material from blood by methods known in the art (e.g., centrifugation, filtration, and the like). As used herein, “peripheral blood plasma” refers to plasma obtained from peripheral blood samples.

“Serum” as used herein includes the fraction of plasma obtained after plasma or blood is permitted to clot and the clotted fraction is removed.

The terms “nucleic acid” is meant to include polymeric form of nucleotides of any length, which contain deoxyribonucleotides, ribonucleotides, and analogs in any combination. Nucleic acids may have three-dimensional structure, and may perform any function, known or unknown. The term nucleic acid includes double-stranded, single-stranded, partially double-stranded, hairpin and triple-helical molecules. Unless otherwise specified or required, any embodiment of the invention described herein that is a nucleic acid encompasses both the double stranded form and each of two complementary forms known or predicted to make up the double stranded form of either the DNA, RNA or hybrid molecule. Nucleic acid may be amplified, recombinant, or may be directly isolated from natural sources. Nucleic acid may include nucleic acid that has been amplified (e.g., using polymerase chain reaction). Specific examples of nucleic acids include a gene or gene fragment, genomic DNA, RNA including mRNA, tRNA, and rRNA, ribozymes, cDNA, recombinant nucleic acids, branched nucleic acids, plasmids, and vectors. Nucleic acids may be natural or synthetic.

The term “genomic nucleic acid” as used herein refers to the nucleic acid in a cell that is present in the cell chromosome(s) of an organism which contains the genes that encode the various proteins of the cells of that organism. A preferred type of genomic nucleic acid is that present in the nucleus of a eukaryotic cell. In a preferred embodiment a genomic nucleic acid is DNA. Genomic nucleic acid can be double stranded or single stranded, or partially double stranded, or partially single stranded or a hairpin molecule. Genomic nucleic acid may be intact or fragmented (e.g., digested with restriction endonucleases or by sonication or by applying shearing force by methods known in the art). In some cases, genomic nucleic acid may include sequence from all or a portion of a single gene or from multiple genes, sequence from one or more chromosomes, or sequence from all chromosomes of a cell. As is well known, genomic nucleic acid includes gene coding regions, introns, 5′ and 3′ untranslated regions, 5′ and 3′ flanking DNA and structural segments such as telomeric and centromeric DNA, replication origins, and intergenic DNA. Genomic nucleic acid representing the total nucleic acid of the genome is referred to as “total genomic nucleic acid.”

Genomic nucleic acid may be obtained by methods of extraction/purification from acellular body fluids as is well known in the art. The ultimate source of genomic nucleic acid can be normal cells or may be cells that contain one or more mutations in the genomic nucleic acid, e.g., duplication, deletion, translocation, and transversion. Included in the meaning of genomic nucleic acid is genomic nucleic acid that has been subjected to an amplification step that increases the amount of the target sequence of interest sought to be detected relative to other nucleic acid sequences in the genomic nucleic acid.

As used herein, the term “cDNA” refers to complementary or copy polynucleotide produced from an RNA template by the action of RNA-dependent DNA polymerase activity (e.g., reverse transcriptase). cDNA can be single stranded, double stranded or partially double stranded. cDNA may contain unnatural nucleotides. cDNA can be modified after being synthesized. cDNA may comprise a detectable label.

The term “isolated” as used herein in context of a polynucleotide or polypeptide refer to a molecule that is substantially separated from the cellular macromolecules with which it is naturally associated. A molecule is isolated if it represents in the composition at least 25%, 50%, 75%, 90%, 95%, or 99% of the cellular macromolecules with which it is naturally associated.

A “gene” refers to a DNA sequence that comprises control and coding sequences necessary for the production of an RNA, which may have a non-coding function (e.g., a ribosomal or transfer RNA) or which may include a polypeptide or a polypeptide precursor. The RNA or polypeptide may be encoded by a full length coding sequence or by any portion of the coding sequence so long as the desired activity or function is retained.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. “Wild-type” may also refer to the sequence at a specific nucleotide position or positions, or the sequence at a particular codon position or positions, or the sequence at a particular amino acid position or positions. As used herein, “mutant” “modified” or “polymorphic” refers to a gene or gene product which displays modifications in sequence and or functional properties (i.e., altered characteristics) when compared to the wild-type gene or gene product. “mutant” “modified” or “polymorphic” also refers to the sequence at a specific nucleotide position or positions, or the sequence at a particular codon position or positions, or the sequence at a particular amino acid position or positions.

A “mutation” is meant to encompass at least a nucleotide variation in a nucleotide sequence relative to the normal sequence. A mutation may include a substitution, a deletion, an inversion or an insertion. With respect to an encoded polypeptide, a mutation may be “silent” and result in no change in the encoded polypeptide sequence or a mutation may result in a change in the encoded polypeptide sequence. For example, a mutation may result in a substitution in the encoded polypeptide sequence. A mutation may result in a frameshift with respect to the encoded polypeptide sequence.

The term “homology” or “homologous” refers to a degree of identity. There may be partial homology or complete homology. A partially homologous sequence is one that has less than 100% sequence identity when compared to another sequence.

“Heterozygous” refers to having different alleles at one or more genetic loci in homologous chromosome segments. As used herein “heterozygous” may also refer to a sample, a cell, a cell population or an organism in which different alleles at one or more genetic loci may be detected. Heterozygous samples may also be determined via methods known in the art such as, for example, nucleic acid sequencing. For example, if a sequencing electropherogram shows two peaks at a single locus and both peaks are roughly the same size; the sample may be characterized as heterozygous. Or, if one peak is smaller than another, but is at least about 25% the size of the larger peak, the sample may be characterized as heterozygous. In some embodiments, the smaller peak is at least about 15% of the larger peak. In other embodiments, the smaller peak is at least about 10% of the larger peak. In other embodiments, the smaller peak is at least about 5% of the larger peak. In other embodiments, a minimal amount of the smaller peak is detected.

“Nucleic acid” or “nucleic acid sequence” as used herein refers to an oligonucleotide, nucleotide or polynucleotide, and fragments or portions thereof, which may be single or double stranded, and represent the sense or antisense strand. A nucleic acid may include DNA or RNA, and may be of natural or synthetic origin and may contain deoxyribonucleotides, ribonucleotides, or nucleotide analogs in any combination.

Non-limiting examples of polynucleotides include a gene or gene fragment, genomic DNA, exons, introns, mRNA, tRNA, rRNA, ribozymes, cDNA, recombinant polynucleotides, branched polynucleotides, plasmids, vectors, isolated DNA of any sequence, isolated RNA of any sequence, synthetic nucleic acid, nucleic acid probes and primers. Polynucleotides may be natural or synthetic. Polynucleotide may comprise modified nucleotides, such as methylated nucleotides and nucleotide analogs, uracyl, other sugars and linking groups such as fluororibose and thiolate, and nucleotide branches. A nucleic acid may be modified such as by conjugation, with a labeling component. Other types of modifications included in this definition are caps, substitution of one or more of the naturally occurring nucleotides with an analog, and introduction of chemical entities for attaching the polynucleotide to other molecules such as proteins, metal ions, labeling components, other polynucleotides or a solid support. Nucleic acid may include nucleic acid that has been amplified (e.g., using polymerase chain reaction).

A fragment of a nucleic acid generally contains at least about 15, 20, 25, 30, 35, 40, 45, 50, 75, 100, 200, 300, 400, 500, 1000 nucleotides or more. Larger fragments are possible and may include about 2,000, 2,500, 3,000, 3,500, 4,000, 5,000 7,500, or 10,000 bases.

The term “specific hybridization” refers to a hybridization interaction between two nucleic acid sequences that share a high degree of complementarity, wherein the hybridization is to the exclusion of hybridization between the nucleic acid of interest and other related nucleic acids. Specific hybridization complexes form under permissive annealing conditions and remain hybridized after any subsequent washing steps. Permissive conditions for annealing of nucleic acid sequences are routinely determinable by one of ordinary skill in the art and may occur, for example, at 65° C. in the presence of about 6×SSC. Stringency of hybridization may be expressed, in part, with reference to the temperature under which the wash steps are carried out. Such temperatures are typically selected to be about 5° C. to 20° C. lower than the thermal melting point (Tm) for the specific sequence at a defined ionic strength and pII. The Tm is the temperature (under defined ionic strength and pH) at which 50% of the target sequence hybridizes to a perfectly matched probe. Equations for calculating Tm and conditions for nucleic acid hybridization are known in the art.

The term “stringent hybridization conditions” as used herein refers to hybridization conditions at least as stringent as the following: hybridization in 50% formamide, 5×SSC, 50 mM NaH₂PO₄, pH 6.8, 0.5% SDS, 0.1 mg/mL sonicated salmon sperm DNA, and 5× Denhart's solution at 42° C. overnight; washing with 2×SSC, 0.1% SDS at 45° C.; and washing with 0.2×SSC, 0.1% SDS at 45° C. In another example, stringent hybridization conditions should not allow for hybridization of two nucleic acids which differ over a stretch of 20 contiguous nucleotides by more than two bases.

Oligonucleotides used as primers or probes for specifically amplifying (i.e., amplifying a particular target nucleic acid sequence) or specifically detecting (i.e., detecting a particular target nucleic acid sequence) a target nucleic acid generally are capable of specifically hybridizing to the target nucleic acid.

As used herein, a “primer” for amplification is an oligonucleotide that is complementary to a target nucleotide sequence that is capable of acting as a point of initiation of synthesis when placed under conditions in which primer extension is initiated (e.g., primer extension associated with an application such as PCR) and leads to addition of nucleotides to the 3′-end of the primer in the presence of a DNA or RNA polymerase. In preferred embodiments, the 3′-nucleotide of the primer is complementary to the target sequence at a corresponding nucleotide position for optimal expression and amplification. A “primer” may occur naturally, as in a purified restriction digest or may be produced synthetically. The term “primer” as used herein includes all forms of primers that may be synthesized including peptide nucleic acid primers, locked nucleic acid primers, phosphorothioate modified primers, labeled primers, and the like. Primers are typically at least about 10, 15, 20, 25, 30, 35, 40, 45, 50, or more nucleotides in length. An optimal length for a particular primer application may be readily determined in the manner described in H. Erlich, PCR Technology, Principles and Application for DNA Amplification (1989).

A “probe” refers to a nucleic acid that interacts with a target nucleic acid via hybridization. Probes may be oligonucleotides, artificial chromosomes, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may comprise modified nucleobases and modified sugar moieties. A probe may be fully complementary to a target nucleic acid sequence or partially complementary. A probe may be used to detect the presence or absence of a target nucleic acid. A probe or probes can be used, for example to detect the presence or absence of a mutation in a nucleic acid sequence by virtue of the sequence characteristics of the target. Probes can be labeled or unlabeled, or modified in any of a number of ways well known in the art. A probe may specifically hybridize to a target nucleic acid. Probes are typically at least about 10, 15, 20, 25, 30, 35, 40, 50 nucleotides or more in length. In preferred embodiments, an NPM1 probe specifically hybridizes to a nucleic acid comprising at least 20 nucleotides that are substantially identical to a region of SEQ ID NO: 1 which encompasses nucleotide positions 1018 and 1019. Preferably, the probe specifically hybridizes to either the wildtype NPM1 sequence or an NPM1 sequence comprising an insertion mutation.

The term “detectable label” as used herein refers to a molecule or a compound or a group of molecules (e.g., a detection system) used to identify a target molecule of interest. Typically, detectable labels represent a component of a detection system and are attached to another molecule that specifically binds to the target molecule. In some cases, the detectable label may be detected directly. In other cases, the detectable label may be a part of a binding pair, which can then be subsequently detected. Signals from the detectable label may be detected by various means and will depend on the nature of the detectable label. Examples of means to detect detectable label include but are not limited to spectroscopic, photochemical, biochemical, immunochemical, electromagnetic, radiochemical, or chemical means, such as fluorescence, chemifluoresence, or chemiluminescence, or any other appropriate means.

The term “target nucleic acid” and “target sequence” are used interchangeably herein and refer to nucleic acid sequence which is intended to be identified. Target sequence can be DNA or RNA. “Target sequence” may be genomic nucleic acid. Target sequences may include wild type sequences, nucleic acid sequences containing point mutations, deletions or duplications, sequence from all or a portion of a single gene or from multiple genes, sequence from one or more chromosomes, or any other sequence of interest. Target sequences may represent alternative sequences or alleles of a particular gene. Target sequence can be double stranded or single stranded, or partially double stranded, or partially single stranded or a hairpin molecule. Target sequence can be about 1-5 bases, about 10 bases, about 20 bases, about 50 bases, about 100 bases, or about 500 bases, or more.

The term “amplification” or “amplify” as used herein includes methods for copying a target nucleic acid, thereby increasing the number of copies of a selected nucleic acid sequence. Amplification may be exponential or linear. A target nucleic acid may be either DNA or RNA. The sequences amplified in this manner form an “amplicon” or “amplification product”. While the exemplary methods described hereinafter relate to amplification using the polymerase chain reaction (PCR), numerous other methods are known in the art for amplification of nucleic acids (e.g., isothermal methods, rolling circle methods, etc.). The skilled artisan will understand that these other methods may be used either in place of, or together with, PCR methods. See, e.g., Saiki, “Amplification of Genomic DNA” in PCR Protocols (1990), Innis et al., Eds., Academic Press, San Diego, Calif., pp 13-20; Wharam, et al., Nucleic Acids Res. (2001), June 1; 29(11):E54-E54; Hafner, et al., Biotechniques (2001), 4:852-6, 858, 860.

As used herein, the term “about” means in quantitative terms, plus or minus 10%.

As used herein the term “normal karyotype” means cells having no chromosomal aberrations which include but not limited to translocations, inversions, and presence of extra chromosomal elements such as microsatellite DNA.

The term “zygosity status” as used herein refers to a sample, a cell population, or an organism as appearing heterozygous, homozygous, or hemizygous as determined by testing methods known in the art and described herein. The term “zygosity status of a nucleic acid” means determining whether the source of nucleic acid appears heterozygous, homozygous, or hemizygous. The “zygosity status” may refer to differences in a single nucleotide in a sequence. In some methods, the zygosity status of a sample with respect to a single mutation may be categorized as homozygous wild-type, heterozygous (i.e., one wild-type allele and one mutant allele), homozygous mutant, or hemizygous (i.e., a single copy of either the wild-type or mutant allele). Because direct sequencing of plasma or cell samples as routinely performed in clinical laboratories does not reliably distinguish between hemizygosity and homozygosity, in some embodiments, these classes are grouped. For example, samples in which no or a minimal amount of wild-type nucleic acid is detected are termed “hemizygous/homozygous mutant.”

The phrase “determining a prognosis” as used herein refers to the process in which the course or outcome of a condition in a patient is predicted. The term “prognosis” does not refer to the ability to predict the course or outcome of a condition with 100% accuracy. Instead, the term refers to identifying an increased or decreased probability that a certain course or outcome will occur in a patient exhibiting a given condition/marker, when compared to those individuals not exhibiting the condition. The nature of the prognosis is dependent upon the specific disease and the condition/marker being assessed. For example, a prognosis may be expressed as the amount of time a patient can be expected to survive, the likelihood that the disease goes into remission, or to the amount of time the disease can be expected to remain in remission.

### BRIEF DESCRIPTION OF THE FIGURES

FIG. 1. is a schematic representation of the Nucleophosmin (NPM1) gene and of Nucleophosmin protein, the gene product of NPM1. The terms “NES”, “NLS”, and “NoLS” indicate nuclear export signal, nuclear localization signal, and nucleolar localization signal in the Nucleophosmin protein respectively. “**” indicates site of mutations in NPM1 gene and Nucleophosmin protein.

FIG. 2A shows the nucleotide sequences of various NPM-1 mutations in exon 12 that have been identified in AML patients. The NPM1 mutant sequences are shown relative to the wildtype (“WT”) NPM1 sequence. FIG. 2B shows the a portion of the nucleolar localization signal (beginning with amino acid 286 of SEQ ID NO: 2) of Nucleophosmin proteins resulting from the mutations identified in FIG. 2A.

FIG. 3. shows the results of detecting a NPM1 mutation present in bone marrow cells, plasma and peripheral blood cells. Panel A represents a size analysis of PCR amplification products from peripheral blood cells (PB cells; top), bone marrow cells (BM cells; middle), and peripheral blood plasma (bottom) from a single AML patient. WT NPM1 (212 bp) is present in each sample type, while a mutant NPM1 containing a 4 bp insertion (216 bp) is only detected in bone marrow and plasma. Left most peak represents a 200 bp standard. Panel B represents a sequence analysis of the mutation of NPM1 in a heterozygous patient as compared to a WT patient. The insertion site is outlined in black, indicating the start of a frameshift in the resulting RNA (as read from right to left from the insertion point).

FIG. 4. indicates the result of NPM1 mutations by size analysis. Analyses were performed on AML patient plasma. The results reveal a novel 4 bp deletion mutant. Size analysis of PCR amplification products distinguishes between WT NPM1 (212 bp; bottom), previously described 4 bp insertion mutants (216 bp; middle), and a novel 4 bp deletion mutant of NPM1 (208 bp; top). Left most peak represents a 200 bp standard.

FIG. 5. indicates the correlation of the presence of the NPM1 insertion mutation and improved clinical outcome of AML patients. NPM1 mutation confers a significant survival advantage in AML patients who are slow to respond to therapy. The Kaplan-Meier plot gives patient survival in weeks as a proportion of the population of AML patients who took more than 35 days to demonstrate a response to therapy. The plot compares NPM1 mutant-positive and mutant-negative patients, showing a significant survival advantage for patients carrying the NPM1 mutation (P=0.027). (E, total events; N, number died).

FIG. 6 provides the cDNA sequence of the human NPM1 gene (SEQ ID NO: 1).

FIG. 7 provides the amino acid sequence of nucleophosmin (SEQ ID NO: 2).

FIG. 8 provides the cDNA sequence of FLT3 (SEQ ID NO: 5).

### DETAILED DESCRIPTION OF THE INVENTION

The present invention is based on the discovery that mutations in the NPM1 gene, which underlie several hematological malignancies, may be reliably detected using nucleic acids isolated from an acellular body fluid (e.g., serum or plasma) obtained from a patient. In particular, it has been discovered that peripheral blood plasma is a reliable sample type for the detection of NPM1 mutations in patients with AML. When bone marrow cells and plasma were tested side by side, there was complete concordance in the paired samples. Furthermore, plasma analysis demonstrated greater sensitivity than NPM1 mutation analysis using peripheral blood cells.

Without wishing to be bound by any theory, it is believe that the high turnover rate of tumor cells as compared with normal cells underlies the increased sensitivity of NPM1 mutation detection plasma relative to peripheral blood cells. Because of this turnover, tumor cells pour into circulation their DNA, RNA and protein, all of which can be substrates for testing. In hematologic malignancies such as AML and MDS, the bulk of the tumor cells are in the bone marrow. However, only relatively few leukemic cells circulate in peripheral blood in some patients, therefore, peripheral blood analysis proved to be unreliable for detecting the NPM1 mutations in some patients.

Plasma and/or serum testing is advantageous because it contains nucleic acid derived from bone marrow tumor cells. Moreover, testing of the plasma serum minimizes the contribution of residual normal cells to the measurements obtained, thus helping to avoid the underestimation sometimes caused by “dilution” of malignant bone marrow samples by lingering normal cells. It is believed that this is due to the fact that, in the plasma, the debris created by the programmed cell death of normal cells is promptly removed by the reticuloendothelial system, while the detritus resulting from the turnover of leukemic cells is far less efficiently eliminated.

As described herein, plasma proved to be more sensitive than peripheral blood cells, with 8% of the cell-based tests yielding false negative results. The false negative results in peripheral blood cells might be attributed to predominantly bone marrow disease without circulating leukemic cells, while the plasma contained genetic material from malignant cells that may have died in the bone marrow and thus gave positive results. Additionally, plasma provides the same ease of collection as peripheral blood cells, avoiding the need for painful and invasive harvesting of bone marrow samples.

To further confirm the clinical value of testing plasma, the mutation results were correlated with clinical observations similar to those reported when bone marrow testing was performed. There was significant correlation of better survival in NPM1 mutation-positive patients who had intermediate cytogenetics and required more than 35 days of treatment to achieve remission. This observation indicates that those AML patients who survive 35 days of therapy without showing signs of remission should not be considered as high risk if they harbor the NPM1 mutation.

The Nucleophosmin Gene (NPM1)

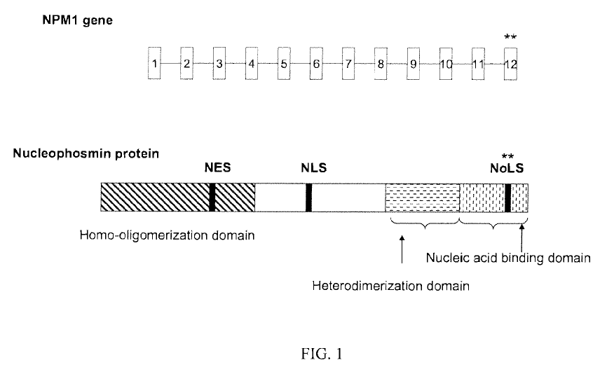

Heterozygous mutation of the nucleophosmin gene (NPM1) has recently been described as one of the most frequent genetic lesions in acute myeloid leukemia (AML). The NPM1 gene is located on chromosome 5q35 in humans. It contains 12 exons. A schematic representation of NPM1 gene is shown in FIG. 1. Exemplary sequence of the genomic DNA comprising NPM1 gene can be found in NCBI GenBank accession number NW_{—}001838954. Sequence of which is incorporated herein by reference.

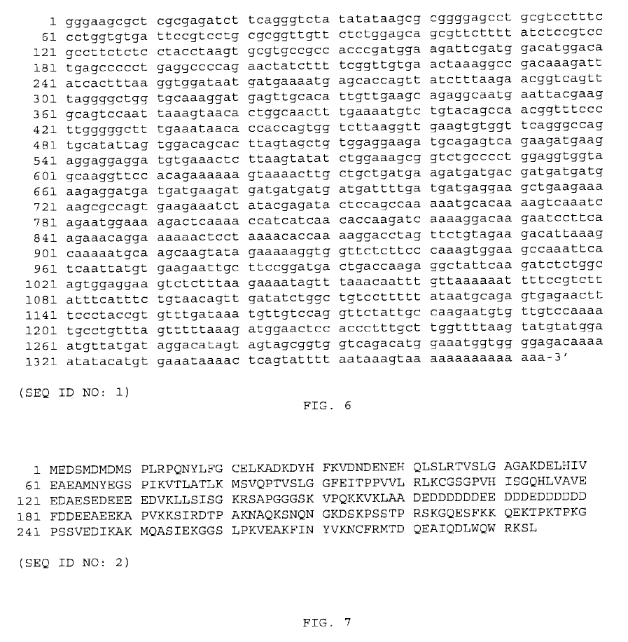

Several variants of NPM1 mRNA are known in the art. Many of the known sequences are full length cDNA sequences and some are partial cDNA sequences. Exemplary NPM1 cDNA sequences include but are not limited to: NCBI GenBank accession numbers: NM_{—}002520, NM_{—}199185 NM_{—}001037738, BC002398, BC050628, BC021983, BC021668, BC016824, BC016768, BC016716, BC014349, BC012566, BC008495, DQ303464, BC009623, BC003670, AY740640, AY740639, AY740638, AY740637, AY740636, AY740635, AY740634, M28699. Sequence of all NPM1 variants indicated above are incorporated herein by reference. One exemplary cDNA sequence of NPM1 gene is provided in SEQ ID NO: 1 (FIG. 6).

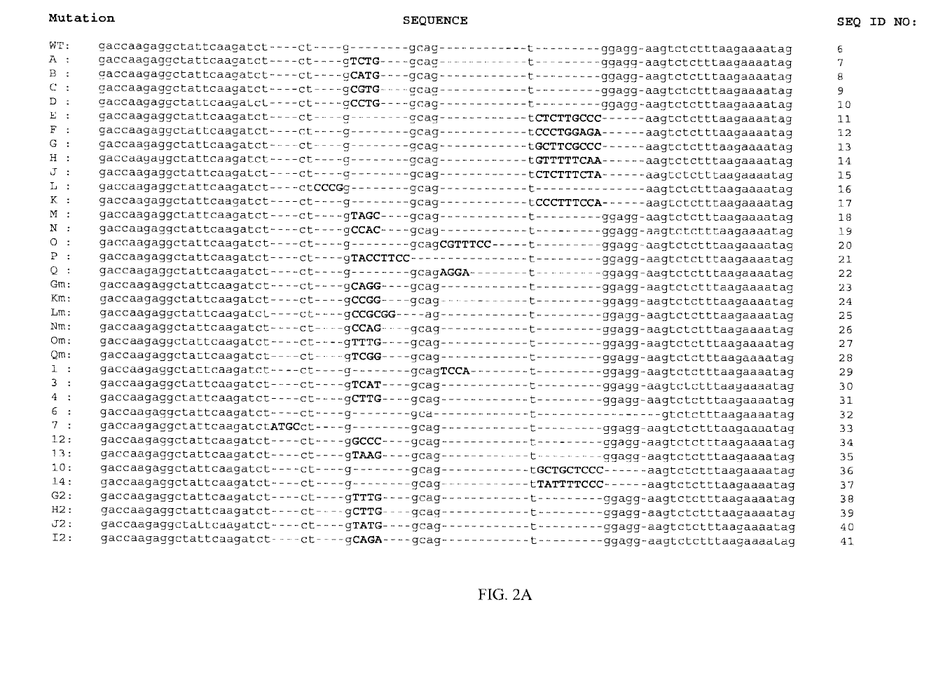

The most common NPM1 mutations that have been identified in AML are 1- or 2-tetranucleotide insertions, a 4-base pair (bp) or 5-bp deletion combined with a 9-bp insertion, and a 9-bp deletion combined with a 14-bp insertion. Majority of these mutations are located in exon 12 and are shown in FIG. 2A.

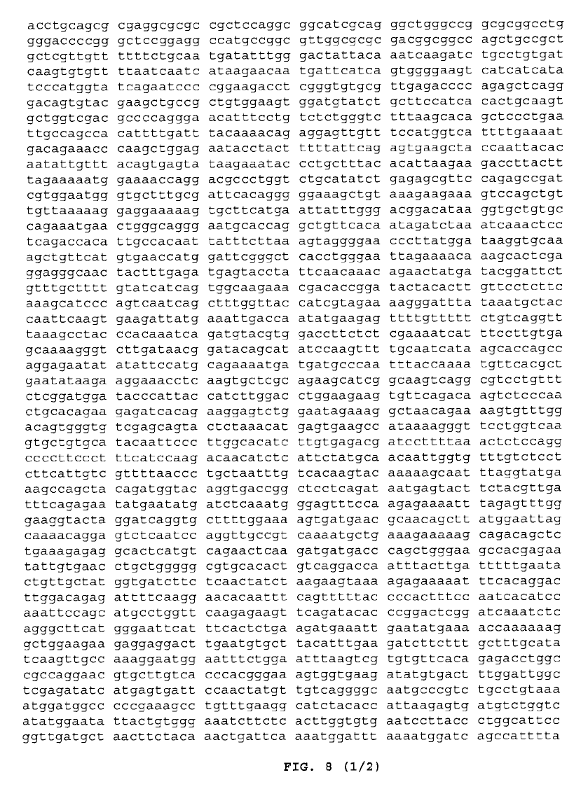

NPM1 exists in two alternatively spliced isoforms. B23.1, the prevalent isoform is present in all tissues and contains 294 amino acids, whereas B23.2, a truncated protein, lacks the last 35 C-terminal amino acids of B23.1 and is expressed at very low levels. The NPM1 molecule (schematically shown in FIG. 1) contains distinct functional domains including an N-terminal homo-oligomerization domain required for formation of NPM dimers and hexamers, a heterodimerization domain implicated in targeting other proteins, such as nucleolin and cyclin-dependent kinase inhibitor p14/alternative reading frame (p14ARF, hereafter referred to as ARF), and a C-terminal nucleic acid-binding domain essential for association with RNA involved in ribosomal RNA processing. The amino acid sequence of the B23.1 NPM1 isoform is provided in SEQ ID NO: 2 (FIG. 7).

Although most NPM1 resides in the nucleolus, it shuttles from the nucleus to cytoplasm. The Nuclear Localization Signal (NLS) drives NPM1 from the cytoplasm to the nucleoplasm, where it is translocated to the nucleolus through its nucleolar localization signal (NoLS). Particularly important residues in NoLS are tryptophan 288 and tryptophan 290 residues of SEQ ID NO: 2. NPM1 remains in nucleoli, even though it contains highly conserved hydrophobic leucine-rich Nuclear Export Signal (NES) motifs within residues 94-102 and 42-49 of SEQ ID NO: 2, which drives it out of the nucleus.

One of the most distinctive features of NPM1 mutants is their aberrant localization in the cytoplasm of leukemic cells. This is causally related to two alterations at the leukemic mutant C-terminus: (i) generation of an additional leucine-rich NES motif; and (ii) loss of tryptophan residues at one or both of positions 288 and 290 of SEQ ID NO: 2 which are crucial for NPM1 nucleolar localization. Mutation of both tryptophans is associated with the very common NES motif, L-xxx-V-xx-V-x-L; retention of tryptophan 288 is associated with rare NES variants in which valine at the second position is replaced by leucine, phenylalanine, cysteine or methionine (Falini et al. Blood. 2006;107: 4514-23). Majority of the NPM1 mutants share the last 5 amino acid residues VSLRK.

NPM1-mutations in AML are often associated with normal cytogenetics, and FLT3 gene internal tandem duplications (FLT3-ITD). Various studies have shown that within the group of AML patients who have a normal karyotype, patients with NPM1 mutation had a complete remission rate similar to or significantly higher than that of patients with wild-type NPM1 AML (Boissel et al. Blood. 2005;106: 3618-3620; Falini et al. N. Engl. J. Med. 2005; 352: 254 266; Suzuki et al. Blood. 2005;106: 2854-2861; Dohner et al. Blood. 2005;106: 3740-6).

Most studies have shown a statistical trend toward favorable outcome in event-free survival and overall survival. Further analyses in the context of other molecular aberrations have shown that patients with NPM1 mutations without concomitant fms-related tyrosine kinase 3 internal tandem duplication (FLT3-ITD) have even a more favorable prognosis than AML patients with FLT3-ITD and has been associated with an approximately 60% probability of survival at 5 years in younger patients (Dohner et al. Blood. 2005; 106: 3740-6).

The FLT3 Gene

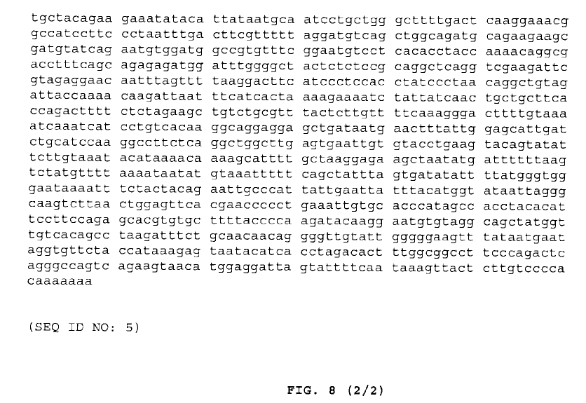

FLT3 gene is located on chromosome 13 in humans. Exemplary sequence of FLT3 gene in human chromosome is disclosed in NCBI GenBank accession number NG_{—}007066, hereby incorporated by reference. The exemplary cDNA sequence of the FLT3 gene is shown in FIG. 8.

In some embodiments the inventions provide methods for detection of FLT3 gene alone or simultaneously with NPM1 in acellular body fluids. In preferred embodiments, the inventions provides methods for detection of one or more mutation of FLT3 gene alone or simultaneously with detection of one or mutation in NPM1 gene.

Biological Sample Collection and Preparation

The methods and compositions of this invention may be used to detect mutations in the NPM1 nucleic acids (e.g., genomic DNA and/or RNA) using a biological sample obtained from an individual. The nucleic acid may be isolated from the sample according to any methods well known to those of skill in the art. If necessary the sample may be collected or concentrated by centrifugation and the like. The cells of the sample may be subjected to lysis, such as by treatments with enzymes, heat, surfactants, ultrasonication, or a combination thereof in order to prepare an acellular fluid. Alternatively, mutations in the NPM1 gene may be detected using an acellular bodily fluid according to the methods described in U.S. Patent Publication US 2007/0248961, hereby incorporated by reference.

Plasma or Serum Preparation Methods

Methods of plasma and serum preparation are well known in the art. Either “fresh” blood plasma or serum, or frozen (stored) and subsequently thawed plasma or serum may be used. Frozen (stored) plasma or serum should optimally be maintained at storage conditions of −20 to −70 degrees centigrade until thawed and used. “Fresh” plasma or serum should be refrigerated or maintained on ice until used, with nucleic acid (e.g., RNA, DNA or total nucleic acid) extraction being performed as soon as possible. Exemplary methods are described below.

Blood may be drawn by standard methods into a collection tube, preferably siliconized glass, either without anticoagulant for preparation of serum, or with EDTA, sodium citrate, heparin, or similar anticoagulants for preparation of plasma. The preferred method if preparing plasma or serum for storage, although not an absolute requirement, is that plasma or serum be first fractionated from whole blood prior to being frozen. This reduces the burden of extraneous intracellular RNA released from lysis of frozen and thawed cells which might reduce the sensitivity of the amplification assay or interfere with the amplification assay through release of inhibitors to PCR such as porphyrins and hematin. “Fresh” plasma or serum may be fractionated from whole blood by centrifugation, using preferably gentle centrifugation at 300-800 times gravity for five to ten minutes, or fractionated by other standard methods. High centrifugation rates capable of fractionating out apoptotic bodies should be avoided. Since heparin may interfere with RT-PCR, use of heparinized blood may require pretreatment with heparinase, followed by removal of calcium prior to reverse transcription. Imai, H., et al., J. Virol. Methods 36:181-184, (1992). Thus, EDTA is the preferred anticoagulant for blood specimens in which PCR amplification is planned.

Nucleic Acid Extraction and Amplification

Optionally, the nucleic acid of the acellular fluid may be amplified in order to facilitate mutation analysis.

Various methods of extraction are suitable for isolating the DNA or RNA. Suitable methods include phenol and chloroform extraction. See Maniatis et al., Molecular Cloning, A Laboratory Manual, 2d, Cold Spring Harbor Laboratory Press, page 16.54 (1989). Numerous commercial kits also yield suitable DNA and RNA including, but not limited to, QIAamp™ mini blood kit, Agencourt Genfind™, Roche Cobas® Roche MagNA Pure® or phenol:chloroform extraction using Eppendorf Phase Lock Gel®, and the NucliSens extraction kit (Biomericux, Marcy l'Etoile, France). In other methods, mRNA may be extracted from patient blood/bone marrow samples using MagNA Pure LC mRNA HS kit and Mag NA Pure LC Instrument (Roche Diagnostics Corporation, Roche Applied Science, Indianapolis, Ind.).

Nucleic acid extracted from tissues, cells, plasma or serum can be amplified using nucleic acid amplification techniques well know in the art. Many of these amplification methods can also be used to detect the presence of mutations simply by designing oligonucleotide primers or probes to interact with or hybridize to a particular target sequence in a specific manner. By way of example, but not by way of limitation these techniques can include the polymerase chain reaction (PCR) reverse transcriptase polymerase chain reaction (RT-PCR), nested PCR, ligase chain reaction. See Abravaya, K., et al., Nucleic Acids Research 23:675-682, (1995), branched DNA signal amplification, Urdea, M. S., et al., AIDS 7 (suppl 2):S11-S14, (1993), amplifiable RNA reporters, Q-beta replication, transcription-based amplification, boomerang DNA amplification, strand displacement activation, cycling probe technology, isothermal nucleic acid sequence based amplification (NASBA). See Kievits, T. et al., J Virological Methods 35:273-286, (1991), Invader Technology, or other sequence replication assays or signal amplification assays.

Serum and plasma RNA is sensitive, specific, and abundant, and may be used instead of (genomic) DNA-based testing. RNA may be extracted from plasma or serum using silica particles, glass beads, or diatoms, as in the method or adaptations of Boom, R., et al., J. Clin. Micro. 28:495-503, (1990). Application of the method adapted by Cheung, R. C., et al., J. Clin Micro. 32:2593-2597, (1994), is described.

For example, size fractionated silica particles are prepared by suspending 60 grams of silicon dioxide (SiO₂, Sigma Chemical Co., St. Louis, Mo.) in 500 milliliters of demineralized sterile double-distilled water. The suspension is then settled for 24 hours at room temperature. Four-hundred thirty (430) milliliters of supernatant is removed by suction and the particles are resuspended in demineralized, sterile double-distilled water added to equal a volume of 500 milliliters. After an additional 5 hours of settlement, 440 milliliters of the supernatant is removed by suction, and 600 microliters of HCl (32% wt/vol) is added to adjust the suspension to a pH2. The suspension is aliquotted and stored in the dark.

Lysis buffer is prepared by dissolving 120 grams of guinidine thiocyanate (GuSCN, Fluka Chemical, Buchs, Switzerland) into 100 milliliters of 0.1 M Tris hydrochloride (Tris-HCl) (pH 6.4), and 22 milliliters of 0.2 M EDTA, adjusted to pH 8.0 with NaOH, and 2.6 grams of Triton X-100 (Packard Instrument Co., Downers Grove, Ill.). The solution is then homogenized. Washing buffer is prepared by dissolving 120 grams of guinidine thiocyanate (GuSCN) into 100 milliliters of 0.1 M Tris-HCl (pH 6.4).

One hundred microliters to two hundred fifty microliters (with greater amounts required in settings of minimal disease) of plasma or serum are mixed with 40 microliters of silica suspension prepared as above, and with 900 microliters of lysis buffer, prepared as above, using an Eppendorf 5432 mixer over 10 minutes at room temperature. The mixture is then centrifuged at 12,000×g for one minute and the supernatant aspirated and discarded. The silica-RNA pellet is then washed twice with 450 microliters of washing buffer, prepared as above. The pellet is then washed twice with one milliliter of 70% (vol/vol) ethanol. The pellet is then given a final wash with one milliliter of acetone and dried on a heat block at 56 degrees centigrade for ten minutes. The pellet is resuspended in 20 to 50 microliters of diethyl procarbonate-treated water at 56 degrees centigrade for ten minutes to elute the RNA. The sample can alternatively be eluted for ten minutes at 56 degrees centigrade with a TE buffer consisting of 10 millimolar Tris-HCl, one millimolar EDTA (pH 8.0) with an RNase inhibitor (RNAsin, 0.5 U/microliter, Promega), with or without Proteinase K (100 ng/ml) as described by Boom, R., et al., J. Clin. Micro. 29:1804-1811, (1991). Following elution, the sample is then centrifuged at 12,000×g for three minutes, and the RNA containing supernatant recovered.

As an alternative method, RNA may be extracted from plasma or serum using the Acid Guanidinium Thiocyanate-Phenol-chloroform extraction method described by Chomczynski, P. and Sacchi, N., Analytical Biochemistry 162:156-159, (1987), or the modified method as described by Chomczynski, P., Biotech 15:532-537, (1993), each of which is hereby incorporated by reference.

Circulating extracellular DNA, including tumor-derived extracellular DNA, is also present in plasma and serum. Since this DNA will additionally be extracted to varying degrees during the RNA extraction methods described above, it may be desirable or necessary (depending upon clinical objectives) to further purify the RNA extract and remove trace DNA prior to proceeding to further RNA analysis. This may be accomplished using DNase, for example by the method as described by Rashtchian, A., PCR Methods Applic. 4:S83-S91, (1994).

Alternatively, primers for further RNA analysis may be constructed which favor amplification of the RNA products, but not of contaminating DNA, such as by using primers which span the splice junctions in RNA, or primers which span an intron. Alternative methods of amplifying RNA but not the contaminating DNA include the methods as described by Moore, R. E., et al., Nucleic Acids Res. 18:1921, (1991), and methods as described by Buchman, G. W., et al., PCR Methods Applic. 3:28-31, (1993), which employs a dU-containing oligonucleotide as an adaptor primer.

It may be desirable to extract RNA, but analyze DNA because of the relative instability of RNA during routine processing and analyses. An isolated RNA sequence may be reproduced as DNA using reverse transcription, which may be performed according to previously published procedures. Various reverse transcriptases may be used, including, but not limited to, MMLV RT, RNase H mutants of MMLV RT such as Superscript and Superscript II (Life Technologies, GIBCO BRL, Gaithersburg, Md.), AMV RT, and thermostable reverse transcriptase from Thermus Thermophilus. For example, one method, but not the only method, which may be used to convert RNA extracted from plasma or serum to cDNA is the protocol adapted from the Superscript II Preamplification system (Life Technologies, GIBCO BRL, Gaithersburg, Md.; catalog no. 18089-011), as described by Rashtchian, A., PCR Methods Applic. 4:S83-S91, (1994).

Mutation Detection

Nucleic acid (e.g., total nucleic acid) may be extracted and amplified from a patient's biological sample using any appropriate method. The amplified product may then be purified, for example by gel purification, and the resulting purified product may be sequenced. Nucleic acid sequencing methods are known in the art; an exemplary sequencing method includes the ABI Prism BigDye Terminator v3.1 Cycle Sequencing Kit (Applied Biosystems, Foster City, Calif.). The sequencing data may then be analyzed for the presence or absence of one or more mutations in the target nucleic acid (e.g., the NPM1 or FLT3 nucleic acid). The sequencing data may also be analyzed to determine the proportion of wild-type to mutant nucleic acid present in the sample.

An alternative method of amplification or mutation detection is allele specific PCR (ASPCR). ASPCR which utilizes matching or mismatching between the template and the 3′ end base of a primer well known in the art. See e.g., U.S. Pat. No. 5,639,611.

Another method of mutation detection is nucleic acid sequencing. Sequencing can be performed using any number of methods, kits or systems known in the art. One example is using dye terminator chemistry and an ABI sequencer (Applied Biosystems, Foster City, Calif.). Sequencing also may involve single base determination methods such as single nucleotide primer extension (“SNapShot” sequencing method) or allele or mutation specific PCR.

In other embodiments, target nucleic acid mutations may be assessed by hybridization of polynucleotide probes which optionally comprise a detectable label. The probe may be detectably labeled by methods known in the art. Useful labels include, for example, fluorescent dyes (e.g., Cy5®, Cy3®, FITC, rhodamine, lanthamide phosphors, Texas red, FAM, JOE, Cal Fluor Red 610®, Quasar 670®), radioisotopes (e.g., ³²P, ³⁵S, ³H, ¹⁴C, ¹²⁵I, ¹³¹I), electron-dense reagents (e.g., gold), enzymes (e.g., horseradish peroxidase, beta-galactosidase, luciferase, alkaline phosphatase), calorimetric labels (e.g., colloidal gold), magnetic labels (e.g., Dynabeads™), biotin, dioxigenin, or haptens and proteins for which antisera or monoclonal antibodies are available. Other labels include ligands or oligonucleotides capable of forming a complex with the corresponding receptor or oligonucleotide complement, respectively. The label can he directly incorporated into the nucleic acid to be detected, or it can be attached to a probe (e.g., an oligonucleotide) or antibody that hybridizes or binds to the nucleic acid to be detected.

In other embodiments, the probes are TaqMan® probes, molecular beacons, and Scorpions (e.g., Scorpion™ probes). These types of probes are based on the principle of fluorescence quenching and involve a donor fluorophore and a quenching moiety. The term “fluorophore” as used herein refers to a molecule that absorbs light at a particular wavelength (excitation frequency) and subsequently emits light of a longer wavelength (emission frequency). The term “donor fluorophore” as used herein means a fluorophore that, when in close proximity to a quencher moiety, donates or transfers emission energy to the quencher. As a result of donating energy to the quencher moiety, the donor fluorophore will itself emit less light at a particular emission frequency that it would have in the absence of a closely positioned quencher moiety.

The term “quencher moiety” as used herein means a molecule that, in close proximity to a donor fluorophore, takes up emission energy generated by the donor and either dissipates the energy as heat or emits light of a longer wavelength than the emission wavelength of the donor. In the latter case, the quencher is considered to be an acceptor fluorophore. The quenching moiety can act via proximal (i.e., collisional) quenching or by Förster or fluorescence resonance energy transfer (“FRET”). Quenching by FRET is generally used in TaqMan® probes while proximal quenching is used in molecular beacon and Scorpion™ type probes. Suitable quenchers are selected based on the fluorescence spectrum of the particular fluorophore. Useful quenchers include, for example, the Black Hole™ quenchers BHQ-1, BHQ-2, and BHQ-3 (Biosearch Technologies, Inc.), and the ATTO-series of quenchers (ATTO 540Q, ATTO 580Q, and ATTO 612Q; Atto-Tec GmbH).

TaqMan® probes (Heid, et al., Genome Res 6: 986-994, 1996) use the fluorogenic 5′ exonuclease activity of Taq polymerase to measure the amount of target sequences in cDNA samples. TaqMan® probes are oligonucleotides that contain a donor fluorophore usually at or near the 5′ base, and a quenching moiety typically at or near the 3′ base. The quencher moiety may be a dye such as TAMRA or may be a non-fluorescent molecule such as 4-(4-dimethylaminophenylazo)benzoic acid (DABCYL). See Tyagi, et al., 16 Nature Biotechnology 49-53 (1998). When irradiated, the excited fluorescent donor transfers energy to the nearby quenching moiety by FRET rather than fluorescing. Thus, the close proximity of the donor and quencher prevents emission of donor fluorescence while the probe is intact.

TaqMan® probes are designed to anneal to an internal region of a PCR product. When the polymerase (e.g., reverse transcriptase) replicates a template on which a TaqMan® probe is bound, its 5′ exonuclease activity cleaves the probe. This ends the activity of the quencher (no FRET) and the donor fluorophore starts to emit fluorescence which increases in each cycle proportional to the rate of probe cleavage. Accumulation of PCR product is detected by monitoring the increase in fluorescence of the reporter dye (note that primers are not labeled). If the quencher is an acceptor fluorophore, then accumulation of PCR product can be detected by monitoring the decrease in fluorescence of the acceptor fluorophore.

With Scorpion primers, sequence-specific priming and PCR product detection is achieved using a single molecule. The Scorpion primer maintains a stem-loop configuration in the unhybridized state. The fluorophore is attached to the 5′ end and is quenched by a moiety coupled to the 3′ end, although in suitable embodiments, this arrangement may be switched The 3′ portion of the stem also contains sequence that is complementary to the extension product of the primer. This sequence is linked to the 5′ end of a specific primer via a non-amplifiable monomer. After extension of the primer moiety, the specific probe sequence is able to bind to its complement within the extended amplicon thus opening up the hairpin loop. This prevents the fluorescence from being quenched and a signal is observed. A specific target is amplified by the reverse primer and the primer portion of the Scorpion primer, resulting in an extension product. A fluorescent signal is generated due to the separation of the fluorophore from the quencher resulting from the binding of the probe element (e.g., the JAK2 probe) of the Scorpion primer to the extension product.

The zygosity status and the ratio of wild-type to mutant nucleic acid in a sample may be determined by methods known in the art including sequence-specific, quantitative detection methods. Other methods may involve determining the area under the curves of the sequencing peaks from standard sequencing electropherograms, such as those created using ABI Sequencing Systems, (Applied Biosystems, Foster City, Calif.). For example, the presence of only a single peak such as a “G” on an electropherogram in a position representative of a particular nucleotide is an indication that the nucleic acids in the sample contain only one nucleotide at that position, the “G.” The sample may then be categorized as homozygous because only one allele is detected. The presence of two peaks, for example, a “G” peak and a “T” peak in the same position on the electropherogram indicates that the sample contains two species of nucleic acids; one species carries the “G” at the nucleotide position in question, the other carries the “T” at the nucleotide position in question. The sample may then be categorized as heterozygous because more than one allele is detected.

The sizes of the two peaks may be determined (e.g, by determining the area under each curve), and a ratio of the two different nucleic acid species may be calculated. A ratio of wild-type to mutant nucleic acid may be used to monitor disease progression, determine treatment, or to make a diagnosis. For example, the number of cancerous cells carrying a specific mutation may change during the course of the disease or therapy. If a base line ratio is established early in the disease, a later determined higher ratio of mutant nucleic acid relative to wild-type nucleic acid may be an indication that the disease is becoming worse or a treatment is ineffective; the number of cells carrying the mutation may be increasing in the patient. A lower ratio of mutant relative to wild-type nucleic acid may be an indication that a treatment is working or that the disease is not progressing; the number of cells carrying the mutation may be decreasing in the patient.

In certain embodiments, the NPM1 nucleic acid comprises and insertion or a deletion mutation. These mutations may conveniently be identified by determining the size of at least a portion of the NPM1 nucleic acid isolated from the patient. Methods for detecting the presence or amount of differently-sized polynucleotides are well known in the art and any of them can be used in the methods described herein. The size separation/detection technique used should permit resolution of nucleic acid as long as they differ from one another by at least one nucleotide. The separation can be performed under denaturing or under non-denaturing or native conditions—i.e., separation can be performed on single- or double-stranded nucleic acids. It is preferred that the separation and detection permits detection of length differences as small as one nucleotide. It is further preferred that the separation and detection can be done in a high-throughput format that permits real time or contemporaneous determination of nucleic acid abundance in a plurality of reaction aliquots taken during the cycling reaction. Useful methods for the separation and analysis of the amplified products include, but are not limited to, electrophoresis (e.g., agarose gel electrophoresis, capillary electrophoresis (CE)), chromatography (HPLC), and mass spectrometry.

In one embodiment, CE is a preferred separation means because it provides exceptional separation of the polynucleotides in the range of at least 10-1,000 base pairs with a resolution of a single base pair. CE can be performed by methods well known in the art, for example, as disclosed in U.S. Pat. Nos. 6,217,731; 6,001,230; and 5,963,456, which are incorporated herein by reference. High-throughput CE apparatuses are available commercially, for example, the HTS9610 High throughput analysis system and SCE 9610 fully automated 96-capillary electrophoresis genetic analysis system from Spectrumedix Corporation (State College, Pa.); P/ACE 5000 series and CEQ series from Beckman Instruments Inc (Fullerton, Calif.); and ABI PRISM 3100 genetic analyzer (Applied Biosystems, Foster City, Calif.). Near the end of the CE column, in these devices the amplified DNA fragments pass a fluorescent detector which measures signals of fluorescent labels. These apparatuses provide automated high throughput for the detection of fluorescence-labeled PCR products.

The employment of CE in the methods described herein permits higher productivity compared to conventional slab gel electrophoresis. By using a capillary gel, the separation speed is increased about 10 fold over conventional slab-gel systems.

With CE, one can also analyze multiple samples at the same time, which is essential for high-throughput. This is achieved, for example, by employing multi-capillary systems. In some instances, the detection of fluorescence from DNA bases may be complicated by the scattering of light from the porous matrix and capillary walls. However, a confocal fluorescence scanner can be used to avoid problems due to light scattering (Quesada et al., Biotechniques (1991), 10:616-25).

In some embodiments, nucleic acid may be analyzed and detected by size using agarose gel electrophoresis. Methods of performing agarose gel electrophoresis are well known in the art. See Sambrook et al., Molecular Cloning: A Laboratory Manual (2nd Ed.) (1989), Cold Spring Harbor Press, N.Y.

In one embodiment, detection is by Southern blotting and hybridization with a labeled probe. The techniques involved in Southern blotting are well known to those of skill in the art and may be found in many standard books on molecular protocols. See Sambrook et al., (1989). Briefly, amplification products are separated by gel electrophoresis. The gel is then contacted with a membrane, such as nitrocellulose, permitting transfer of the nucleic acid and non-covalent binding. Subsequently, the membrane is incubated with a chromophore-conjugated probe that is capable of hybridizing with a target amplification product. Detection is by exposure of the membrane to x-ray film or ion-emitting detection devices.

### EXAMPLES

### Example 1

### NPM1 Mutation Detection in Plasma-Derived Nucleic Acids

In order to assess the viability of plasma as a source of genetic material for mutational analysis, bone marrow cells, peripheral blood cells and peripheral blood plasma from 31 newly diagnosed patients with AML were analyzed simultaneously for NPM1 mutations and results were compared between the three sample types.

Genomic DNA was extracted from patient bone marrow or whole blood samples using the BioRobot EZl Blood DNA kit (Qiagen, Valencia, Calif.). Total nucleic acid was extracted from plasma using the NucliSens extraction kit on the EasyMag system (bioMerieux, Durham, N.C.). The NPM1 gene PCR amplification for all sample t)yes was performed using a NPM1 forward primer that hybridizes to NPM1 intron 11 and reverse primer that hybridizes to a NPM1 exon 12. The forward and reverse primers are labeled with 6-carboxyfluorescein (6-FAM; Eurogentec, San Diego, Calif.). The NPM1 mutated or wildtype alleles were verified by determining the size of PCR products using the ABI3100 Genetic Analyzer (Applied Biosystems, Foster City, Calif.). The sequences of the forward and reverse primers are given below.

(SEQ ID NO: 3)

| | |
|---|---|
| Forward primer: | 5′-tta act ctc tgg tgg tag aat |
| | gaa-3′ |

(SEQ ID NO: 4)

| | |
|---|---|
| Reverse primer: | 5′-tgt tac aga aat gaa ata aga |
| | cgg-3′ |

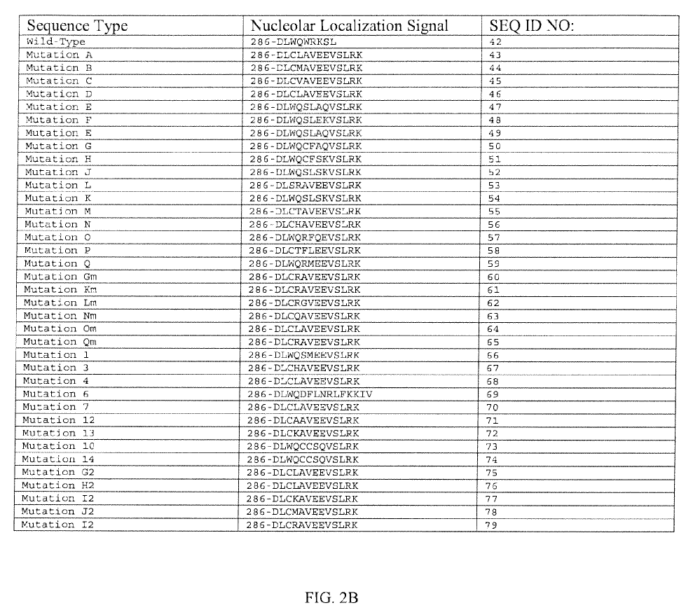

Using these amplification primers, wildtype (WT) NPM1 nucleic acids displayed a 212 bp peak, while NPM1 insertion mutants displayed an extra 216 bp peak in addition to the NPM1 WT peak (see, for example, FIG. 3A). FIG. 3B demonstrates that the four base insertion/frameshift mutation is also capable of being identified in a heterozygous patient compared to a wildtype, and that the mutation results from a CTCT insertion. These results demonstrate that the foregoing method is robust and capable of distinguishing between wildtype and the 4 bp insertion NPM1 mutant nucleic acid isolated from all sources tested, including bone marrow cells, peripheral blood cells, and plasma.

The plasma from the 31 patients showed complete concordance with bone marrow cells, but a discrepancy with peripheral blood cells was observed. Mutated NPM1 nucleic acid was detected in 6 of the 31 paired peripheral blood plasma and bone marrow cell samples. However, when peripheral blood cells were assessed, one of the six samples containing mutated NPM-1 nucleic acid (as assessed in bone marrow cells and plasma), was incorrectly identified as containing wild-type NPM-1 using peripheral blood cell analysis. (FIG. 3A). In this single patient mutation assessment of NPM1 nucleic acid in peripheral blood cells proved inaccurate, but assessment using bone marrow cell and plasma samples showed unmistakable mutation via insertion. In further support of the accuracy of plasma-based testing for NPM1 mutations, no mutation-positive peripheral blood or bone marrow cell samples gave a false negative when the plasma was assayed. These data validated the use of a plasma-based assay for detection of NPM1 mutations in plasma nucleic acid samples.

### Example 2

### NPM1 Mutations in AML and MDS Patients and the Identification of a Novel Mutation

NPM1 nucleic acid analysis was performed on randomly collected pairs of plasma and peripheral blood cells samples from AML (98 samples) and myclodysplastic syndrome (MDS) (28 samples) patients treated at the University of Texas, MD Anderson Cancer Center. All samples were collected from previously untreated patients before therapy was initiated. All MDS patients were off any kind of therapy at the time of obtaining samples for analysis. All samples were collected using Institutional Review Board-approved protocols, all patients provided informed consent, and the study conformed to the code of ethics of the World Medical Association (Declaration of Helsinki). Clinical data were collected by chart review and were part of the leukemia database at MD Anderson Cancer Center. Diagnosis was based on complete morphologic, immunophenotypic, cytogenetic and molecular analysis and classification was according to French American British (FAB) classification. All patients with MDS required evidence of dysplasia in at least two lineages. Cytogenetic status was classified as favorable (t(15;17), t(8,21), or inv16)), unfavorable (−5, −7 or complex (≧3) abnormalities), or intermediate (all others). Performance status (PS), determined with the Zubrod scoring system, was categorized as good (0 or 1) or bad (2-4). Responders are patients who achieved complete response (CR), according to the International Working group criteria for CR.

The characteristics of the AML and MDS patients included in this study are listed in Table 1. The median age was 62 (range, 18 to 82) for the AML patients and 68 (range, 43 to 81) for the MDS patients. Most of the AML patients had either intermediate (34%) or poor (59%) cytogenetic status. Half of the MDS patients were classified as having refractory anemia with increased blasts in transformation (RAEB-T), and 46% had refractory anemia with increased blasts (RAEB) according to the French-American-British (FAB) classification. Only 3% of patients had acute progranulocytic leukemia (APL), while 24% had leukemia with monocytoid differentiation (Table 1). Classification of the AML and MDS patients was based on the FAB classification rather than the World Health Organization (WHO) classification.

TABLE 1
Characteristics of AML and MDS patients

| Characteristics | AML (n = 98) | | MDS (n = 28) | |
|---|---|---|---|---|
| Age, median (range) | 62 | (18-82) | 68 | (43-81) |
| WBC count, median × 10⁹/mL | 8.9 | (0.9-183.6) | 2.4 | (0.8-45.4) |
| (range) | | | | |
| Hemoglobin, median g/dL | 7.9 | (3.8-13) | 8.3 | (3.5-10.7) |
| (range) | | | | |
| Platelets, median × 10⁹/mL | 45 | (7-245) | 42.5 | (10-222) |
| (range) | | | | |

| | | |
|---|---|---|
| Zubrod Performance Status | | |
| 0-1 (%) | 65 | 84 |
| 2-4 (%) | 35 | 16 |
| Cytogenetics | | |
| Intermediate (%) | 34 | 61 |
| Favorable (%) | 7 | 0 |
| Unfavorable (%) | 59 | 39 |
| FAB Classification | | |
| M0-2 (%) | 70 | — |
| M3 (%) | 3 | — |
| M4/M5 (%) | 24 | — |
| M6/M7 (%) | 3 | — |
| RA (%) | — | 0 |
| RAEB (%) | — | 46 |
| RAEB-T (%) | — | 50 |
| CMML (%) | — | 4 |

Mutations in NPM1 nucleic acid were detected in 24 (24%) of the 98 AML plasma samples, while only 22 (22%) of the cell samples revealed the mutation (Table 2). Therefore, 8% of the positive samples gave a false negative result when peripheral blood cells were analyzed. The two AML cases for which the NPM1 mutation was detected in the plasma DNA, but not the peripheral blood cell DNA were characterized as having no circulating blast cells. However, the failure detect NPM1 mutations in AML peripheral blood lacking blast cells was not universal. NPM1 mutations were detected in peripheral blood cell DNA in other cases for which no circulating blast cells were reported.

TABLE 2
NPM1 mutation frequency in AML and MDS patients

| | | |
|---|---|---|
| FAB classification | Peripheral blood cells | Plasma |

AML Patients: NPM1 Mutation Percent Positivity

| M0/M1/M2 (n = 68) | 10% | (n = 10) | 12% | (n = 12) |
|---|---|---|---|---|
| M3 (n = 3) | 0 | | 0 | |
| M4/M5 (n = 24) | 50% | (n = 12) | 50% | (n = 12) |
| M6/M7 (n = 3) | 0 | | 0 | |
| Totals (n = 98) | 22% | (n = 22) | 24% | (n = 24) |

MDS Patients: NPM1 Mutation Percent Positivity

| RA (n = 0) | N/A | | N/A | |
|---|---|---|---|---|
| RAEB (n = 13) | 4% | (n = 1) | 4% | (n = 1) |
| RAEB-T (n = 14) | 0 | (0) | 0 | (0) |
| CMML (n = 1) | 0 | (0) | 0 | (0) |
| Totals (n = 28) | 4% | (n = 1) | 4% | (n = 1) |

The highest rate of NPM1 mutation was detected in AML patients classified as M2 by the FAB classification (38% of M2). Although the number of cases is small, the M4/M5 group also had a high rate of NPM1 mutation (Table 2). In addition to the AML patients, the plasma from 28 previously untreated MDS patients for NPM1 mutations were tested. Of these patients, only 1 patient (4%) was found to harbor a NPM1 mutation. This MDS patient with NPM1 mutation had RAEB (Table 2), but with relatively limited cytopenia (white blood count of 4.5×10⁹/mL), which suggests the possibility of early leukemia rather than myelodysplastic disease. All patients were classified according to FAB classification. If the World Health Organization (WHO) classification was used, all the RAEB-T would have been classified as AML and the prevalence of NPM1 mutation would have been 21% instead of 24%. The lack of NPM1 mutations in patients with RAEB-T supports the concept that these cases possess characteristics more consistent with MDS than with acute leukemia.

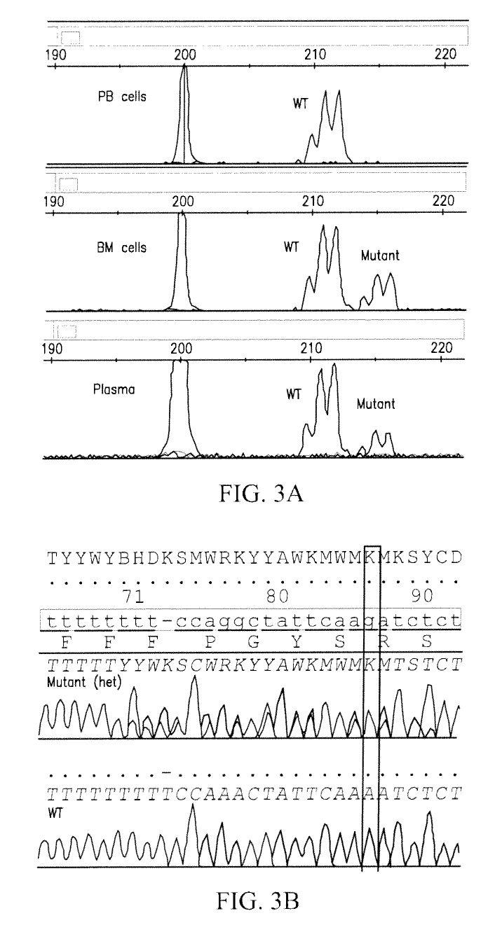

In most patients the NPM1 mutation comprised the 4 bp insertion of CTCT as shown in FIG. 3. In a single patient, a novel 4 base deletion was detected in exon 12 of NPM1 (FIG. 4). This patient had acute progranulocytic leukemia (APL) and expressed the short form of the RARα-PML fusion transcript, and responded to therapy.

### Example 3

### Clinical and Pathologic Characteristics of AML Patients in the Presence or Absence of NPM1 Mutations

The 98 AML patient samples characterized in Example 2 were further characterized based on their hematological make-up. Patients with NPM1 mutations were found to have a significantly higher white blood cell (WBC) count as compared with patients lacking the mutation (Table 3). These patients also had a higher percentage of blasts in peripheral blood and bone marrow. In addition, the blasts in patients with mutated NPM1 expressed significantly lower levels of HLADR, CD13 and CD34, and significantly higher levels of CD33 (Table 3).

TABLE 3
Comparison of clinical and pathologic characteristics of AML patients
in the presence or absence of NPM1 mutations

| | NPM1 Mutation (−) | | NPM1 Mutation (+) | | |
|---|---|---|---|---|---|
| | (n = 74) | | (n = 24) | | |
| Characteristic | Median | (Range) | Median | (Range) | P value* |
| Blasts-Periph. | 23 | (0-97) | 61 | (0-99) | 0.002 |
| Blood (%) | | | | | |
| Blasts-Bone | 42 | (5-97) | 72 | (22-98) | 0.002 |
| Marrow (%) | | | | | |
| HLA-DR (%) | 91 | (1-99) | 69 | (0-98) | 0.005 |
| CD13 (%) | 92 | (2-100) | 68 | (10-97) | 0.02 |
| CD34 (%) | 86 | (0-100) | 1 | (0-54) | 0.0001 |
| CD33 (%) | 94 | (2-100) | 99 | (68-100) | 0.0004 |
| WBC count | 6.7 | (0.9-161) | 24.4 | (1.1-183) | 0.0009 |
| (cells/ml) | | | | | |

*Two-tailed Student's t-test

There was also a difference between NPM1-mutated and WT NPM1 AML-patients with respect to their cytogenetic abnormalities and the presence of a mutation in the FLT3 gene (Table 4). Generally, patients having the NPM1 mutation were associated with a better cytogenetic profile; 12% having poor cytogeneics versus 41% in of the wildtype NPM1 group.

TABLE 4
Cytogenetics and FLT3 mutation status in AML patients in the presence or
absence of NPM1 mutation

| | NPM1 | NPM1 |
|---|---|---|
| | Mutation (−) | Mutation (+) |
| Cytogenetics | (n = 74) | (n = 24) |
| Good [inversion 16, t(8; 21), | 8% (n = 6) | 0 |
| t(15; 17)] | | |
| Poor (−5, −7, or complex) | 41% (n = 30) | 12% (n = 3) |
| Intermediate (other cytogenetics, | 51% (n = 38) | 88% (n = 21) |
| including diploidy) | | |
| FLT3 Mutation (+)* | 26% (7 of 27) | 56% (5 of 9) |

*FLT3 mutation data only available for 36 patients.

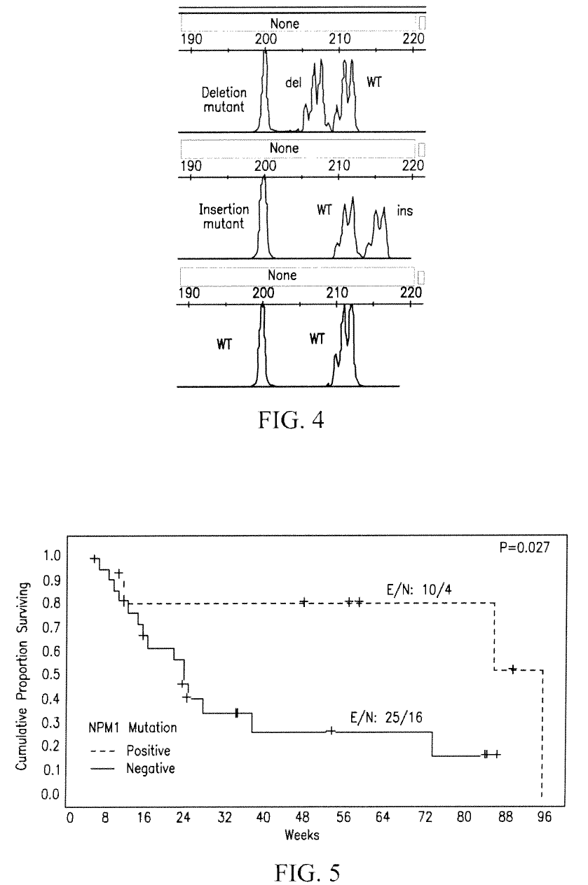

It was further observed that response to therapy was slightly higher in AML patients with the NPM1 mutation than in AML patients without the mutation, although the difference was not quite significant (P=0.06). These data comport with previous, larger studies which demonstrate that the NPM1 mutation is characteristic of AML and indictive of a patient's responsiveness to induction chemotherapy. See, for example, Falini et al., N. Engl. Med (2005) 352:254-266. When all patients were considered there was no significant difference in survival between patients with NPM1 mutation and those without the mutation. However, when considering only patients having intermediate cytogenetics, those with the NPM1 mutation demonstrated a relatively longer event-free survival than patients without the mutation, (P=0.056). The low P-value is possibly due to the small number of patients studied. The most striking difference in survival was found in mutation-positive patients with intermediate cytogenetics who required more than 35 days to respond to therapy. In these patients, survival was significantly longer than in patients lacking the NPM1 mutation (FIG. 5).

All publications, patent applications, patents and other references mentioned herein are expressly incorporated by reference in their entirety, to the same extent as if each were incorporated by reference individually. In case of conflict, the present specification, including definitions, will control.

Although the present inventions have been described with reference to exemplary and alternative embodiments, workers skilled in the art will recognize that changes may be made in form and detail without departing from the spirit and scope of the invention. For example, although different exemplary and alternative embodiments may have been described as including one or more features providing one or more benefits, it is contemplated that the described features may be interchanged with one another or alternatively be combined with one another in the described exemplary embodiments or in other alternative embodiments. Because the technology of the present invention is relatively complex, not all changes in the technology are foreseeable. The present invention described with reference to the exemplary and alternative embodiments and set forth in the following claims is manifestly intended to be as broad as possible. For example, unless specifically otherwise noted, the claims reciting a single particular element also encompass a plurality of such particular elements.

What is claimed is:

1. A method of determining a prognosis of an individual diagnosed with acute myelogenous leukemia (AML), said method comprising
determining the presence or absence of one or more mutations in an NPM1 nucleic acid, wherein said NPM1 nucleic acid is obtained from an acellular body fluid of said individual, and
providing a prognosis for said individual, wherein the presence of one or more mutations in the NPM1 gene is indicative of better prognosis for said individual relative to an individual diagnosed with AML and lacking said one or more mutations.

2. The method of claim 1, wherein said acellular body fluid is serum or plasma.

3. The method of claim 2, wherein the presence or absence of one or more mutations is determined relative to SEQ ID NO: 1.

4. The method of claim 1, wherein said NPM1 nucleic acid is genomic DNA.

5. The method of claim 1, wherein said NPM1 nucleic acid is mRNA.

6. The method of claim 1, wherein one of said mutations in the NPM1 nucleic acid comprises a CTCT insertion.

7. The method of claim 6, wherein said insertion is after the nucleotide corresponding to position 1018 of SEQ ID NO: 1.

8. The method of claim 1, wherein at least one of said mutations in the NPM1 nucleic acid is selected from FIG. 2A or FIG. 2B.

9. The method of claim 1, farther comprising detecting the presence or absence of one or more mutations in FLT3 gene.

10. The method of claim 9, wherein said one or more mutations in FLT3 gene is a duplication of an internal tandem repeat.

11. The method of claim 9, wherein the presence of one or mutation in NPM1 gene and absence of one or more mutation in FLT3 gene is an indicative of better prognosis of said individual diagnosed with AML.

12. The method of claim 1, further comprising determining the cytogenetics of said individual.

13. The method of claim 1, wherein said method comprises amplifying NPM1 nucleic acid obtained from acellular body fluid of said AML patient and hybridizing said amplified NPM1 nucleic acid with an oligonucleotide probe that is capable of specifically detecting the presence of at least NPM1 nucleic acid mutation under hybridization conditions.

14. The method of claim 1, wherein said method comprises determining the size of at least a portion of the NPM1 nucleic acid, wherein an increased size is indicative of the presence of an insertion mutation.

15. The method of claim 1, wherein said prognosis relates to remission rate.

16. The method of claim 1, wherein said prognosis in said AML patient relates to overall survival.

17. A method of determining a prognosis of an individual diagnosed with a AML, said method comprising
determining the presence or absence of an insertion mutation in an NPM1 nucleic acid, wherein said NPM1 nucleic acid is obtained from an acellular body fluid of said individual, and
providing a prognosis for said individual, wherein the presence of said insertion mutation is an indicative of better prognosis for said individual relative to an individual diagnosed with AML and lacking said insertion mutation.

18. The method of claim 17, wherein said insertion mutation comprises a CTCT insertion following the nucleotide corresponding to position 1018 of SEQ ID NO: 1.

19. The method of claim 17, further comprising detecting the presence or absence of one or more mutations in FLT3 gene.

20. The method of claim 19, wherein said one or more mutations in FLT3 gene is a duplication of internal tandem repeat.

21. The method of claim 19, wherein the presence of said insertion mutation and the absence a mutation in the FLT3 gene is an indicative of better prognosis of said individual diagnosed with AML.

22. The method of claim 17, wherein said prognosis relates to remission rate or overall survival.

23. The method of claim 17, wherein said method comprises determining the size of at least a portion of the NPM1 nucleic acid, wherein an increased size is indicative of the presence of an insertion mutation.

24. The method of claim 17, wherein said method comprises amplifying said NPM1 nucleic acid using an amplification primer comprising the sequence of SEQ ID NO: 3 or SEQ ID NO: 4.

25. The method of claim 17, wherein said method comprises amplifying said NPM1 nucleic acid using a pair of amplification primers comprising the sequence of SEQ ID NOs: 3 and 4.

26. A method of diagnosing an individual with a hematological disorder, said method comprising
determining the presence or absence of a translocation in an NPM1 nucleic acid, wherein said NPM1 nucleic acid is obtained from an acellular body fluid of said individual, and
diagnosing said individual with a hematological disorder when a translocation in an NPM1 nucleic acid is detected.

27. The method of claim 26, wherein said hematological disorder is selected from the group consisting of anaplastic large cell lymphoma, acute promyelocytic leukemia, and acute myelogenous leukemia.

28. The method of claim 26, wherein said translocation is between the NPM1 gene an a second gene selected from the group consisting of anaplastic large cell lymphoma kinase, retinoic acid receptor-alpha, and myelodysplasia/myeloid leukemia factor 1.

29. The method of claim 26, comprising further determining the presence or absence of one or more mutations in an NPM1 nucleic acid.

30. The method of claim 29, wherein the presence or absence of one or more mutations is determined relative to SEQ ID NO: 1.

31. The method of claim 30, wherein one of said mutations in the NPM1 nucleic acid comprises a CTCT insertion.

32. The method of claim 31, wherein said insertion is after the nucleotide corresponding to position 1018 of SEQ ID NO: 1.

33. The method of claim 30, wherein at least one of said mutations in the NPM1 nucleic acid is selected from FIG. 2A or FIG. 2B.

34. The method of claim 26, wherein said NPM1 nucleic acid is genomic DNA.

35. The method of claim 26, wherein said NPM1 nucleic acid is mRNA.


SEQUENCE LISTING (86 sequences)

SEQ ID NO: 1 - DNA, 1373 bp, Homo sapiens
gggaagcgctcgcgagatcttcagggtctatatataagcgcggggagcctgcgtcctttccctggtgtgattccgtcctgcgcggttgttctctggagcagcgttcttttatctccgtccgccttctctcctacctaagtgcgtgccgccacccgatggaagattcgatggacatggacatgagccccctgaggccccagaactatcttttcggttgtgaactaaaggccgacaaagattatcactttaaggtggataatgatgaaaatgagcaccagttatctttaagaacggtcagtttaggggctggtgcaaaggatgagttgcacattgttgaagcagaggcaatgaattacgaaggcagtccaattaaagtaacactggcaactttgaaaatgtctgtacagccaacggtttcccttgggggctttgaaataacaccaccagtggtcttaaggttgaagtgtggttcagggccagtgcatattagtggacagcacttagtagctgtggaggaagatgcagagtcagaagatgaagaggaggaggatgtgaaactcttaagtatatctggaaagcggtctgcccctggaggtggtagcaaggttccacagaaaaaagtaaaacttgctgctgatgaagatgatgacgatgatgatgaagaggatgatgatgaagatgatgatgatgatgattttgatgatgaggaagctgaagaaaaagcgccagtgaagaaatctatacgagatactccagccaaaaatgcacaaaagtcaaatcagaatggaaaagactcaaaaccatcatcaacaccaagatcaaaaggacaagaatccttcaagaaacaggaaaaaactcctaaaacaccaaaaggacctagttctgtagaagacattaaagcaaaaatgcaagcaagtatagaaaaaggtggttctcttcccaaagtggaagccaaattcatcaattatgtgaagaattgcttccggatgactgaccaagaggctattcaagatctctggcagtggaggaagtctctttaagaaaatagtttaaacaatttgttaaaaaattttccgtcttatttcatttctgtaacagttgatatctggctgtcctttttataatgcagagtgagaactttccctaccgtgtttgataaatgttgtccaggttctattgccaagaatgtgttgtccaaaatgcctgtttagtttttaaagatggaactccaccctttgcttggttttaagtatgtatggaatgttatgataggacatagtagtagcggtggtcagacatggaaatggtggggagacaaaaatatacatgtgaaataaaactcagtattttaataaagtaaaaaaaaaaaaaaa

SEQ ID NO: 2 - protein, 294 aa, Homo sapiens
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVAVEEDAESEDEEEEDVKLLSISGKRSAPGGGSKVPQKKVKLAADEDDDDDDEEDDDEDDDDDDFDDEEAEEKAPVKKSIRDTPAKNAQKSNQNGKDSKPSSTPRSKGQESFKKQEKTPKTPKGPSSVEDIKAKMQASIEKGGSLPKVEAKFINYVKNCFRMTDQEAIQDLWQWRKSL

SEQ ID NO: 3 - DNA, 24 bp, Artificial Sequence
  Description of Artificial Sequence Synthetic primer
ttaactctctggtggtagaatgaa

SEQ ID NO: 4 - DNA, 24 bp, Artificial Sequence
  Description of Artificial Sequence Synthetic primer
tgttacagaaatgaaataagacgg

SEQ ID NO: 5 - DNA, 3848 bp, Homo sapiens
acctgcagcgcgaggcgcgccgctccaggcggcatcgcagggctgggccggcgcggcctggggaccccgggctccggaggccatgccggcgttggcgcgcgacggcggccagctgccgctgctcgttgttttttctgcaatgatatttgggactattacaaatcaagatctgcctgtgatcaagtgtgttttaatcaatcataagaacaatgattcatcagtggggaagtcatcatcatatcccatggtatcagaatccccggaagacctcgggtgtgcgttgagaccccagagctcagggacagtgtacgaagctgccgctgtggaagtggatgtatctgcttccatcacactgcaagtgctggtcgacgccccagggaacatttcctgtctctgggtctttaagcacagctccctgaattgccagccacattttgatttacaaaacagaggagttgtttccatggtcattttgaaaatgacagaaacccaagctggagaatacctactttttattcagagtgaagctaccaattacacaatattgtttacagtgagtataagaaataccctgctttacacattaagaagaccttactttagaaaaatggaaaaccaggacgccctggtctgcatatctgagagcgttccagagccgatcgtggaatgggtgctttgcgattcacagggggaaagctgtaaagaagaaagtccagctgttgttaaaaaggaggaaaaagtgcttcatgaattatttgggacggacataaggtgctgtgccagaaatgaactgggcagggaatgcaccaggctgttcacaatagatctaaatcaaactcctcagaccacattgccacaattatttcttaaagtaggggaacccttatggataaggtgcaaagctgttcatgtgaaccatggattcgggctcacctgggaattagaaaacaaagcactcgaggagggcaactactttgagatgagtacctattcaacaaacagaactatgatacggattctgtttgcttttgtatcatcagtggcaagaaacgacaccggatactacacttgttcctcttcaaagcatcccagtcaatcagctttggttaccatcgtagaaaagggatttataaatgctaccaattcaagtgaagattatgaaattgaccaatatgaagagttttgtttttctgtcaggtttaaagcctacccacaaatcagatgtacgtggaccttctctcgaaaatcatttccttgtgagcaaaagggtcttgataacggatacagcatatccaagttttgcaatcataagcaccagccaggagaatatatattccatgcagaaaatgatgatgcccaatttaccaaaatgttcacgctgaatataagaaggaaacctcaagtgctcgcagaagcatcggcaagtcaggcgtcctgtttctcggatggatacccattaccatcttggacctggaagaagtgttcagacaagtctcccaactgcacagaagagatcacagaaggagtctggaatagaaaggctaacagaaaagtgtttggacagtgggtgtcgagcagtactctaaacatgagtgaagccataaaagggttcctggtcaagtgctgtgcatacaattcccttggcacatcttgtgagacgatccttttaaactctccaggccccttccctttcatccaagacaacatctcattctatgcaacaattggtgtttgtctcctcttcattgtcgttttaaccctgctaatttgtcacaagtacaaaaagcaatttaggtatgaaagccagctacagatggtacaggtgaccggctcctcagataatgagtacttctacgttgatttcagagaatatgaatatgatctcaaatgggagtttccaagagaaaatttagagtttgggaaggtactaggatcaggtgcttttggaaaagtgatgaacgcaacagcttatggaattagcaaaacaggagtctcaatccaggttgccgtcaaaatgctgaaagaaaaagcagacagctctgaaagagaggcactcatgtcagaactcaagatgatgacccagctgggaagccacgagaatattgtgaacctgctgggggcgtgcacactgtcaggaccaatttacttgatttttgaatactgttgctatggtgatcttctcaactatctaagaagtaaaagagaaaaatttcacaggacttggacagagattttcaaggaacacaatttcagtttttaccccactttccaatcacatccaaattccagcatgcctggttcaagagaagttcagatacacccggactcggatcaaatctcagggcttcatgggaattcatttcactctgaagatgaaattgaatatgaaaaccaaaaaaggctggaagaagaggaggacttgaatgtgcttacatttgaagatcttctttgctttgcatatcaagttgccaaaggaatggaatttctggaatttaagtcgtgtgttcacagagacctggccgccaggaacgtgcttgtcacccacgggaaagtggtgaagatatgtgactttggattggctcgagatatcatgagtgattccaactatgttgtcaggggcaatgcccgtctgcctgtaaaatggatggcccccgaaagcctgtttgaaggcatctacaccattaagagtgatgtctggtcatatggaatattactgtgggaaatcttctcacttggtgtgaatccttaccctggcattccggttgatgctaacttctacaaactgattcaaaatggatttaaaatggatcagccattttatgctacagaagaaatatacattataatgcaatcctgctgggcttttgactcaaggaaacggccatccttccctaatttgacttcgtttttaggatgtcagctggcagatgcagaagaagcgatgtatcagaatgtggatggccgtgtttcggaatgtcctcacacctaccaaaacaggcgacctttcagcagagagatggatttggggctactctctccgcaggctcaggtcgaagattcgtagaggaacaatttagttttaaggacttcatccctccacctatccctaacaggctgtagattaccaaaacaagattaatttcatcactaaaagaaaatctattatcaactgctgcttcaccagacttttctctagaagctgtctgcgtttactcttgttttcaaagggacttttgtaaaatcaaatcatcctgtcacaaggcaggaggagctgataatgaactttattggagcattgatctgcatccaaggccttctcaggctggcttgagtgaattgtgtacctgaagtacagtatattcttgtaaatacataaaacaaaagcattttgctaaggagaagctaatatgattttttaagtctatgttttaaaataatatgtaaatttttcagctatttagtgatatattttatgggtgggaataaaatttctactacagaattgcccattattgaattatttacatggtataattagggcaagtcttaactggagttcacgaaccccctgaaattgtgcacccatagccacctacacattccttccagagcacgtgtgcttttaccccaagatacaaggaatgtgtaggcagctatggttgtcacagcctaagatttctgcaacaacaggggttgtattgggggaagtttataatgaataggtgttctaccataaagagtaatacatcacctagacactttggcggccttcccagactcagggccagtcagaagtaacatggaggattagtattttcaataaagttactcttgtccccacaaaaaaa

SEQ ID NO: 6 - DNA, 56 bp, Homo sapiens
gaccaagaggctattcaagatctctggcagtggaggaagtctctttaagaaaatag

SEQ ID NO: 7 - DNA, 60 bp, Homo sapiens
gaccaagaggctattcaagatctctgtctggcagtggaggaagtctctttaagaaaatag

SEQ ID NO: 8 - DNA, 60 bp, Homo sapiens
gaccaagaggctattcaagatctctgcatggcagtggaggaagtctctttaagaaaatag

SEQ ID NO: 9 - DNA, 60 bp, Homo sapiens
gaccaagaggctattcaagatctctgcgtggcagtggaggaagtctctttaagaaaatag

SEQ ID NO: 10 - DNA, 60 bp, Homo sapiens
gaccaagaggctattcaagatctctgcctggcagtggaggaagtctctttaagaaaatag

SEQ ID NO: 11 - DNA, 60 bp, Homo sapiens
gaccaagaggctattcaagatctctggcagtctcttgcccaagtctctttaagaaaatag

SEQ ID NO: 12 - DNA, 60 bp, Homo sapiens
gaccaagaggctattcaagatctctggcagtccctggagaaagtctctttaagaaaatag

SEQ ID NO: 13 - DNA, 60 bp, Homo sapiens
gaccaagaggctattcaagatctctggcagtgcttcgcccaagtctctttaagaaaatag

SEQ ID NO: 14 - DNA, 60 bp, Homo sapiens
gaccaagaggctattcaagatctctggcagtgtttttcaaaagtctctttaagaaaatag

SEQ ID NO: 15 - DNA, 60 bp, Homo sapiens
gaccaagaggctattcaagatctctggcagtctctttctaaagtctctttaagaaaatag

SEQ ID NO: 16 - DNA, 55 bp, Homo sapiens
gaccaagaggctattcaagatctctcccgggcagtaagtctctttaagaaaatag

SEQ ID NO: 17 - DNA, 60 bp, Homo sapiens
gaccaagaggctattcaagatctctggcagtccctttccaaagtctctttaagaaaatag

SEQ ID NO: 18 - DNA, 60 bp, Homo sapiens
gaccaagaggctattcaagatctctgtagcgcagtggaggaagtctctttaagaaaatag

SEQ ID NO: 19 - DNA, 60 bp, Homo sapiens
gaccaagaggctattcaagatctctgccacgcagtggaggaagtctctttaagaaaatag

SEQ ID NO: 20 - DNA, 63 bp, Homo sapiens
gaccaagaggctattcaagatctctggcagcgtttcctggaggaagtctctttaagaaaatag

SEQ ID NO: 21 - DNA, 60 bp, Homo sapiens
gaccaagaggctattcaagatctctgtaccttcctggaggaagtctctttaagaaaatag

SEQ ID NO: 22 - DNA, 60 bp, Homo sapiens
gaccaagaggctattcaagatctctggcagaggatggaggaagtctctttaagaaaatag

SEQ ID NO: 23 - DNA, 60 bp, Homo sapiens
gaccaagaggctattcaagatctctgcagggcagtggaggaagtctctttaagaaaatag

SEQ ID NO: 24 - DNA, 60 bp, Homo sapiens
gaccaagaggctattcaagatctctgccgggcagtggaggaagtctctttaagaaaatag

SEQ ID NO: 25 - DNA, 60 bp, Homo sapiens
gaccaagaggctattcaagatctctgccgcggagtggaggaagtctctttaagaaaatag

SEQ ID NO: 26 - DNA, 60 bp, Homo sapiens
gaccaagaggctattcaagatctctgccaggcagtggaggaagtctctttaagaaaatag

SEQ ID NO: 27 - DNA, 60 bp, Homo sapiens
gaccaagaggctattcaagatctctgtttggcagtggaggaagtctctttaagaaaatag

SEQ ID NO: 28 - DNA, 60 bp, Homo sapiens
gaccaagaggctattcaagatctctgtcgggcagtggaggaagtctctttaagaaaatag

SEQ ID NO: 29 - DNA, 60 bp, Homo sapiens
gaccaagaggctattcaagatctctggcagtccatggaggaagtctctttaagaaaatag

SEQ ID NO: 30 - DNA, 60 bp, Homo sapiens
gaccaagaggctattcaagatctctgtcatgcagtggaggaagtctctttaagaaaatag

SEQ ID NO: 31 - DNA, 60 bp, Homo sapiens
gaccaagaggctattcaagatctctgcttggcagtggaggaagtctctttaagaaaatag

SEQ ID NO: 32 - DNA, 48 bp, Homo sapiens
gaccaagaggctattcaagatctctggcatgtctctttaagaaaatag

SEQ ID NO: 33 - DNA, 60 bp, Homo sapiens
gaccaagaggctattcaagatctatgcctggcagtggaggaagtctctttaagaaaatag

SEQ ID NO: 34 - DNA, 60 bp, Homo sapiens
gaccaagaggctattcaagatctctggcccgcagtggaggaagtctctttaagaaaatag

SEQ ID NO: 35 - DNA, 60 bp, Homo sapiens
gaccaagaggctattcaagatctctgtaaggcagtggaggaagtctctttaagaaaatag

SEQ ID NO: 36 - DNA, 60 bp, Homo sapiens
gaccaagaggctattcaagatctctggcagtgctgctcccaagtctctttaagaaaatag

SEQ ID NO: 37 - DNA, 60 bp, Homo sapiens
gaccaagaggctattcaagatctctggcagttattttcccaagtctctttaagaaaatag

SEQ ID NO: 38 - DNA, 60 bp, Homo sapiens; identical to SEQ ID NO: 27

SEQ ID NO: 39 - DNA, 60 bp, Homo sapiens; identical to SEQ ID NO: 31

SEQ ID NO: 40 - DNA, 60 bp, Homo sapiens
gaccaagaggctattcaagatctctgtatggcagtggaggaagtctctttaagaaaatag

SEQ ID NO: 41 - DNA, 60 bp, Homo sapiens
gaccaagaggctattcaagatctctgcagagcagtggaggaagtctctttaagaaaatag

SEQ ID NO: 42 - protein, 9 aa, Homo sapiens
DLWQWRKSL

SEQ ID NO: 43 - protein, 13 aa, Homo sapiens
DLCLAVEEVSLRK

SEQ ID NO: 44 - protein, 13 aa, Homo sapiens
DLCMAVEEVSLRK

SEQ ID NO: 45 - protein, 13 aa, Homo sapiens
DLCVAVEEVSLRK

SEQ ID NO: 46 - protein, 13 aa, Homo sapiens
DLCLAVEEVSLRK

SEQ ID NO: 47 - protein, 13 aa, Homo sapiens
DLWQSLAQVSLRK

SEQ ID NO: 48 - protein, 13 aa, Homo sapiens
DLWQSLEKVSLRK

SEQ ID NO: 49 - protein, 13 aa, Homo sapiens
DLWQSLAQVSLRK

SEQ ID NO: 50 - protein, 13 aa, Homo sapiens
DLWQCFAQVSLRK

SEQ ID NO: 51 - protein, 13 aa, Homo sapiens
DLWQCFSKVSLRK

SEQ ID NO: 52 - protein, 13 aa, Homo sapiens
DLWQSLSKVSLRK

SEQ ID NO: 53 - protein, 13 aa, Homo sapiens
DLSRAVEEVSLRK

SEQ ID NO: 54 - protein, 13 aa, Homo sapiens
DLWQSLSKVSLRK

SEQ ID NO: 55 - protein, 13 aa, Homo sapiens
DLCTAVEEVSLRK

SEQ ID NO: 56 - protein, 13 aa, Homo sapiens
DLCHAVEEVSLRK

SEQ ID NO: 57 - protein, 13 aa, Homo sapiens
DLWQRFQEVSLRK

SEQ ID NO: 58 - protein, 13 aa, Homo sapiens
DLCTFLEEVSLRK

SEQ ID NO: 59 - protein, 13 aa, Homo sapiens
DLWQRMEEVSLRK

SEQ ID NO: 60 - protein, 13 aa, Homo sapiens
DLCRAVEEVSLRK

SEQ ID NO: 61 - protein, 13 aa, Homo sapiens
DLCRAVEEVSLRK

SEQ ID NO: 62 - protein, 13 aa, Homo sapiens
DLCRGVEEVSLRK

SEQ ID NO: 63 - protein, 13 aa, Homo sapiens
DLCQAVEEVSLRK

SEQ ID NO: 64 - protein, 13 aa, Homo sapiens
DLCLAVEEVSLRK

SEQ ID NO: 65 - protein, 13 aa, Homo sapiens
DLCRAVEEVSLRK

SEQ ID NO: 66 - protein, 13 aa, Homo sapiens
DLWQSMEEVSLRK

SEQ ID NO: 67 - protein, 13 aa, Homo sapiens
DLCHAVEEVSLRK

SEQ ID NO: 68 - protein, 13 aa, Homo sapiens
DLCLAVEEVSLRK

SEQ ID NO: 69 - protein, 15 aa, Homo sapiens
DLWQDFLNRLFKKIV

SEQ ID NO: 70 - protein, 13 aa, Homo sapiens
DLCLAVEEVSLRK

SEQ ID NO: 71 - protein, 13 aa, Homo sapiens
DLCAAVEEVSLRK

SEQ ID NO: 72 - protein, 13 aa, Homo sapiens
DLCKAVEEVSLRK

SEQ ID NO: 73 - protein, 13 aa, Homo sapiens
DLWQCCSQVSLRK

SEQ ID NO: 74 - protein, 13 aa, Homo sapiens
DLWQCCSQVSLRK

SEQ ID NO: 75 - protein, 13 aa, Homo sapiens
DLCLAVEEVSLRK

SEQ ID NO: 76 - protein, 13 aa, Homo sapiens
DLCLAVEEVSLRK

SEQ ID NO: 77 - protein, 13 aa, Homo sapiens
DLCKAVEEVSLRK

SEQ ID NO: 78 - protein, 13 aa, Homo sapiens
DLCMAVEEVSLRK

SEQ ID NO: 79 - protein, 13 aa, Homo sapiens
DLCRAVEEVSLRK

SEQ ID NO: 80 - protein, 10 aa, Artificial Sequence
  Description of Artificial Sequence Synthetic peptide
LXXXVXXVXL

SEQ ID NO: 81 - protein, 5 aa, Artificial Sequence
  Description of Artificial Sequence Synthetic peptide
VSLRK

SEQ ID NO: 82 - DNA, 29 bp, Homo sapiens
tyywybhdksmwrkyyawkmwmkmksycd

SEQ ID NO: 83 - DNA, 28 bp, Homo sapiens
ttttttttccaggctattcaagatctct

SEQ ID NO: 84 - protein, 9 aa, Homo sapiens
FFFPGYSRS

SEQ ID NO: 85 - DNA, 29 bp, Homo sapiens
tttttyywkscwrkyyawkmwmkmtstct

SEQ ID NO: 86 - DNA, 29 bp, Homo sapiens
tttttttttccaggctattcaagatctct